(19) United States
(12) Patent Application Publication
(10) Pub. No.: US 2017/0204151 A1
(43) Pub. Date: Jul. 20, 2017

(54) Mesenchymal Stem Cells Expressing Biomarkers that Predict the Effectiveness of Mesenchymal Stem Cells for Treating Diseases and Disorders

(71) Applicants: Darwin J. PROCKOP, Philadelphia, PA (US); Ryang Hwa LEE, Temple, TX (US); Ji Min YU, Busan (KR); Joo Youn OH, Seoul (KR); John RENEAU, Rochester, MN (US); Barry BERKOWITZ, Framingham, MA (US); THE TEXAS A&M UNIVERSITY SYSTEM, College Station, TX (US); TEMPLE THERAPEUTICS, INC., Framingham, MA (US)

(72) Inventors: Darwin J. Prockop, Philadelphia, PA (US); Ryang Hwa Lee, Round Rock, TX (US); Ji Min Yu, Busan (KR); Joo Youn Oh, Seoul (KR); John Reneau, Rochester, MN (US); Barry A. Berkowitz, Framingham, MA (US)

(21) Appl. No.: 15/327,890

(22) PCT Filed: Jul. 24, 2015
(86) PCT No.: PCT/US15/42031

§ 371 Date: Jan. 20, 2017

Related U.S. Application Data
(60) Provisional application No. 62/029,662, filed on Jul. 28, 2014.

(51) Int. Cl. C07K 14/525; A61K 35/28; A61K 38/19; C12Q 1/68; C12N 5/0775

(52) U.S. Cl. CPC C07K 14/525; C12Q 1/6881; C12N 5/0663; A61K 38/191; A61K 35/28; C12Q 2600/158; C12N 2501/392; A61K 2035/124

(57) ABSTRACT

Isolated mesenchymal stem cells, which produce mRNA encoding TSG-6 protein or a biologically active fragment, derivative, or analogue thereof in an amount of at least a first preselected amount, or produce mRNA encoding TSG-6 protein or a biologically active fragment, derivative, or analogue thereof in an amount that does not exceed a second preselected amount, as determined by an assay, such as a RT-PCR assay. Isolated mesenchymal stem cells that produce mRNA encoding TSG-6 protein or a biologically active fragment, derivative, or analogue thereof in an amount of at least the first preselected amount are useful in treating diseases, conditions, and disorders associated with inflammation, while isolated mesenchymal stem cells that produce mRNA encoding TSG-6 protein or a biologically active fragment, derivative, or analogue thereof in an amount that does not exceed the second preselected amount are useful in treating bone diseases, conditions, and disorders, including bone injuries.

This application claims priority based on provisional application Ser. No. 62/029,662, filed Jul. 28, 2014, the contents of which are incorporated by reference in their entirety.

This invention relates to mesenchymal stem cells that produce RNA, including but not limited to messenger RNA, or mRNA, encoding certain proteins in amounts that are predictive of the efficacy of such mesenchymal stem cells in treating various diseases and disorders. More particularly, in one non-limiting embodiment, this invention relates to selecting isolated mesenchymal stem cells that produce mRNA encoding anti-inflammatory proteins or inflammation modulatory proteins, such as, for example, tumor necrosis factor-alpha stimulating gene 6 (TSG-6) protein or a biologically active fragment, derivative, or analogue thereof in an amount of at least a preselected amount. Such isolated mesenchymal stem cells are effective in treating a variety of diseases and disorders associated with inflammation.

In another non-limiting embodiment, this invention relates to selecting isolated mesenchymal stem cells that produce mRNA encoding anti-inflammatory proteins or inflammation modulatory proteins, such as TSG-6 protein or a biologically active fragment, derivative, or analogue thereof in an amount that does not exceed a preselected amount. Such isolated mesenchymal stem cells are effective in treating a variety of bone diseases and disorders, as well as bone injuries.

Human mesenchymal stem/progenitor cells (hMSCs) from bone marrow, adipose tissues, placenta, umbilical cord and other tissues currently are being administered to large numbers of patients. Over 80 clinical trials with hMSCs have been registered (http://clinicaltrials.gov), and five have reached the Phase II or III stage of development (Syed and Evans, 2013). The trials are proceeding even though cultures of the cells are heterogeneous, and there is large variability among different preparations of hMSCs, depending on conditions such as differences among donors, conditions used to expand the cells in culture, and random sampling in harvesting the cells from bone marrow and other tissues (Huang et al., 2013; Keating, 2012; Phinney et al., 1999; Prockop and Oh, 2012). The variability among preparations also is confounded by the lack of definitive markers for the cells. In addition, there are no biomarkers to predict the efficacy of hMSC samples in vivo. Therefore, the value of the data obtained from different clinical trials may be compromised by variations in the quality of the hMSCs employed.

Recent data suggest that the therapeutic effects of the cells were explained in part by their paracrine effects such as expression of factors that modulate inflammatory and immune responses, that limit growth of cancers, or that enhance tissue repair (Bernardo and Fibbe, 2013; Keating, 2012; Lee et al., 2011; Prockop and Oh, 2012). It was observed recently that intravenously infused hMSCs modulated excessive sterile inflammation and thereby improved symptoms in mouse models for myocardial infarction (Lee et al., 2009), corneal injury (Roddy et al., 2011) or peritonitis (Choi et al., 2011), in part because the hMSCs were activated to secrete TSG-6, a protein that is a natural modulator of inflammation (Milner and Day, 2003; Wisniewski and Vilcek, 1997; Wisniewski et al., 2005). Of special interest was that a well-known model of chemical injury of the cornea made it possible to obtain quantitative dose-response data for the effectiveness of recombinant TSG-6 on the both neutrophil infiltration and the functional integrity of the tissue (Oh et al., 2010). Using this model, here we demonstrated that bone marrow-derived hMSCs isolated from different donors showed wide variations in their efficacy in modulating inflammation. A biomarker now has been identified that predicts the in vivo efficacy of different donor derived MSCs in suppressing inflammation. The biomarker should prove useful in selecting preparations of mesenchymal stem cells for treating various diseases, disorders, and conditions in patients.

In accordance with an aspect of the present invention, there is provided a composition comprising isolated mesenchymal stem cells that produce mRNA encoding tumor necrosis factor-α gene 6 (TSG-6) protein or a biologically active fragment, derivative, or analogue thereof in an amount of at least a preselected amount, as measured by an assay. The assay comprises assaying the level of mRNA encoding TSG-6 protein or a biologically active fragment, derivative, or analogue thereof that is produced by an isolated population of mesenchymal stem cells. Then, the amount of mRNA encoding TSG-6 protein or a biologically active fragment, derivative, or analogue thereof produced by the population of isolated mesenchymal stem cells is determined, whereby it is determined whether the population of isolated mesenchymal stem cells produce mRNA encoding TSG-6 protein or a biologically active fragment, derivative, or analogue thereof in an amount of at least the preselected amount.

Although the scope of this aspect of the present invention is not intended to be limited to any theoretical reasoning, it is believed that, in general, mesenchymal stem cells that produce increased amounts of mRNA encoding TSG-6 protein or a biologically active fragment, derivative, or analogue thereof also will express TSG-6 protein or a biologically active fragment, derivative, or analogue thereof in increased amounts. Therefore, mesenchymal stem cells that produce mRNA encoding TSG-6 protein or a biologically active fragment, derivative, or analogue thereof in amounts that are at least that of the preselected amount are more likely to express TSG-6 protein or a biologically active fragment, derivative, or analogue thereof in an amount such that the mesenchymal stem cells are useful particularly for treating inflammatory diseases and disorders.

In general, the composition comprising isolated mesenchymal stem cells is prepared by providing a population of mesenchymal stem cells by obtaining a cell population containing the mesenchymal stem cells from a donor, and then isolating or purifying the mesenchymal stem cells from the cell population. For example, in a non-limiting embodiment, a sample of bone marrow cells may be obtained from an animal donor, such as a primate, including human and non-human primates, and the mesenchymal stem cells are isolated or purified from the remainder of the bone marrow cells by means known to those skilled in the art.

In a non-limiting embodiment, the TSG-6 protein encoded by the mRNA is the “native” TSG-6 protein, which has 277 amino acid residues as shown hereinbelow.

MIILIYLFLL LWEDTQGWGF KDGIFHNSIW LERAAGVYHR
EARSGKYKLT YAEAKAVCEF EGGHLATYKQ LEAARKIGFH
VCAAGWMAKG RVGYPIVKPG PNCGFGKTGI IDYGIRLNRS
ERWDAYCYNP HAKECGGVFT DPKQIFKSPG FPNEYEDNQI
CYWHIRLKYG QRIHLSFLDF DLEDDPGCLA DYVEIYDSYD
DVHGFVGRYC GDELPDDIIS TGNVMTLKFL SDASVTAGGF
QIKYVAMDPV SKSSQGKNTS TTSTGNKNFL AGRFSHL

In a non-limiting embodiment, the isolated mesenchymal stem cells have been genetically engineered with a polynucleotide encoding TSG-6 protein or a biologically active fragment, derivative, or analogue thereof.

In a non-limiting embodiment, the isolated mesenchymal stem cells are genetically engineered with a polynucleotide encoding the “native” TSG-6 protein hereinabove described. In another non-limiting embodiment, the isolated mesenchymal stem cells are genetically engineered with a polynucleotide encoding a biologically active fragment, derivative, or analogue of TSG-6 protein.

In another non-limiting embodiment, the TSG-6 protein or biologically active fragment, derivative, or analogue thereof is a fragment of TSG-6 protein known as a TSG-6-LINK protein, or a TSG-6 link module domain. In one non-limiting embodiment, the TSG-6 link module domain consists of amino acid residues 1 through 133 of the above-mentioned sequence.

In another non-limiting embodiment, the TSG-6 link module domain consists of amino acid residues 1 through 98 of the above-mentioned sequence and is described in Day, et al., Protein Expr. Purif., Vol. 8, No. 1, pgs. 1-16 (August 1996).

In another non-limiting embodiment, the TSG-6 protein or a biologically active fragment, derivative, or analogue thereof, has a “His-tag” at the C-terminal thereof. The term “His-tag”, as used herein, means that one or more histidine residues are bound to the C-terminal of the TSG-6 protein or biologically active fragment, derivative, or analogue thereof. In another non-limiting embodiment, the “His-tag” has six histidine residues at the C-terminal of the TSG-6 protein or a biologically active fragment, derivative, or analogue thereof.

In a non-limiting embodiment, when the TSG-6 protein, or biologically active fragment, derivative, or analogue thereof, includes a “His-tag”, at the C-terminal thereof, the TSG-6 protein or biologically active fragment, derivative, or analogue thereof, may include a cleavage site that provides for cleavage of the “His-tag” from the TSG-6 protein or biologically active fragment, derivative, or analogue thereof, after the TSG-6 protein, or biologically active fragment, derivative, or analogue thereof is produced.

The polynucleotide encoding TSG-6 protein or biologically active fragment, derivative or analogue thereof may be in the form of DNA (including but not limited to genomic DNA (gDNA) or cDNA, or RNA. The polynucleotide encoding TSG-6 protein or a biologically active fragment, derivative, or analogue thereof may be contained in an appropriate expression vector, such as an adenoviral vector, adeno-associated virus vector, retroviral vector, or lentiviral vector that is introduced into the mesenchymal stem cells, or may be contained in a transposon that is introduced into the cell, or the polynucleotide may be introduced into the cell as naked DNA or RNA. Such introduction of the polynucleotide may be introduced into the cell by any of a variety of means known to those skilled in the art, such as calcium phosphate precipitation, liposomes, gene guns, or by clustered regularly interspersed short palindromic repeats, or CRISPR, technology.

In another non-limiting embodiment, the polynucleotide encoding TSG-6 protein or a biologically active fragment, derivative, or analogue thereof is introduced into a “safe harbor” chromosomal locus in the mesenchymal stem cells. In a non-limiting embodiment, the safe harbor chromosomal locus is the adeno-associated virus S1 (AAVS1) locus on human chromosome 19. In another non-limiting embodiment, the safe harbor chromosomal locus is located on human chromosome 13.

The isolated mesenchymal stem cells then are assayed for levels of mRNA encoding TSG-6 protein or a biologically active fragment, derivative, or analogue thereof in order to determine whether the isolated mesenchymal stem cells produce mRNA encoding TSG-6 protein or a biologically active fragment, derivative, or analogue thereof in an amount which is at least the preselected amount.

In a non-limiting embodiment, the population of isolated mesenchymal stem cells is assayed for levels of mRNA encoding TSG-6 protein or a biologically active fragment, derivative, or analogue thereof produced by the isolated mesenchymal stem cells by conducting a reverse transcription polymerase chain reaction, or RT-PCR, assay.

In a non-limiting embodiment, the amount of mRNA encoding TSG-6 protein produced by a “standard” or “reference” population of mesenchymal stem cells is determined by a reverse transcription PCR assay. The amount of mRNA encoding TSG-6 protein by the “standard” or “reference” population of mesenchymal stem cells thus is the preselected amount. In a non-limiting embodiment, the “standard” or “reference” population is a population from a human donor known as Donor 7052 or a human donor known as Donor 7075. These cell populations have been found to produce similar amounts of mRNA encoding TSG-6 protein and are available from the institute for Regenerative Medicine, Texas A & M College of Medicine. The amount of mRNA encoding TSG-6 protein or a biologically active fragment, derivative, or analogue thereof produced by a test population of mesenchymal stem cells then is determined by the reverse transcription PCR assay. The test population of mesenchymal stem cells contains approximately the same number of mesenchymal stem cells as the “standard” or “reference” population. When the “standard” or “reference” population of mesenchymal stem cells is from Donor 7052 or Donor 7075 as hereinabove described, if the amount of mRNA encoding TSG-6 protein or a biologically active fragment, derivative, or analogue thereof produced by the test population is a least about 10 times the amount of mRNA encoding TSG-6 protein or a biologically active fragment, derivative, or analogue thereof produced by the “standard” or “reference” population, the mesenchymal stem cells from the test population are considered to be suitable especially for treating inflammatory diseases and disorders.

The isolated mesenchymal stem cells of the present invention, which have been determined by an assay to produce mRNA encoding TSG-6 protein or a biologically active fragment, derivative, or analogue thereof in an amount of at least a preselected amount may be administered in an amount effective to treat an inflammatory disease or disorder in an animal, or treat a disease or disorder associated with inflammation in an animal. In a non-limiting embodiment, the animal is a primate, which includes human and non-human primates.

Inflammatory diseases and disorders, and diseases and disorders associated with inflammation which may be treated with the isolated mesenchymal stem cells selected in accordance with the present invention include, but are not limited to, myocardial infarction, cardiac muscle cell necrosis, atherosclerosis, diseases and disorders of the eye, including, but not limited to, corneal diseases and disorders, including corneal injury, diseases and disorders of the vitrea, diseases and disorders of the retina, age-related macular degeneration, and other diseases and disorders related to sterile inflammation.

The term “sterile inflammation”, as used herein, means inflammation that is not caused by a pathogen (i.e., bacteria, virus, etc.), but which is caused in response to an injury or abnormal stimulation caused by a physical, chemical, or biological molecule (e.g., protein, DNA, etc.). Such reactions include, but are not limited to, the local reactions and resulting morphologic changes, destruction or removal of the injurious material, and responses that lead to repair and healing.

One underlying theme in inflammatory disease is a perturbation of the cellular immune response that results in recognition of proteins, such as host proteins (antigens), as foreign. Thus the inflammatory response becomes misdirected at host tissues with effector cells targeting specific organs or tissues, often resulting in irreversible damage. The self-recognition aspect of autoimmune disease often is reflected by the clonal expansion of T-cell subsets characterized by a particular T-cell receptor (TCR) subtype in the disease state. Often, inflammatory disease also is characterized by an imbalance in the levels of T-helper (Th) subsets (i.e., Th1 cells versus Th2 cells).

Sterile inflammatory diseases and conditions may be systemic (i.e., lupus) or localized to particular tissues or organs.

Examples of sterile inflammatory diseases include, without limitation, myocardial infarction (MI), diabetes, stroke, Alzheimer's disease, multiple sclerosis, parkinsonism, nephritis, cancer, inflammatory diseases involving acute or chronic inflammation of bone and/or cartilage in a joint, anaphylactic reaction, asthma, conjunctivitis, systemic lupus erythematosus, pulmonary sarcoidosis, ocular inflammation, allergy, emphysema, ischemia-reperfusion injury, fibromyalgia and inflammatory cutaneous diseases such as psoriasis and dermatitis, or an arthritis such as rheumatoid arthritis, gouty arthritis, juvenile rheumatoid arthritis, and osteoarthritis.

The isolated mesenchymal stem cells of the present invention, which produce mRNA encoding TSG-6 protein or a biologically active fragment, derivative, or analogue thereof in an amount of at least a preselected amount may be administered topically or systemically, such as, for example, by intravenous, intraarterial, intraperitoneal, intramuscular, or subcutaneous administration. Alternatively, isolated the mesenchymal stem cells may be administered directly to the site(s) of inflammation in the patient.

The isolated mesenchymal stem cells, which produce mRNA encoding TSG-6 protein or a biologically active fragment, derivative, or analogue thereof in an amount of at least a preselected amount are administered in conjunction with an acceptable pharmaceutical carrier or excipient. Such pharmaceutical carriers or excipients include, but, are not limited to, water, saline solution, human serum albumin, oils, polyethylene glycol, or PEG, dextrose, glycerin, propylene glycol, or other synthetic solvents, antiadherents, binders (e.g., starches, sugars, cellulose, modified cellulose such as hydroxyethyl cellulose, hydroxypropyl cellulose, and methyl cellulose, lactose, sugar alcohols such as xylitol, sorbitol and maltitol, gelatin, polyvinyl pyrrolidone, polyethylene glycol), coatings (e.g., shellac, corn protein, zein, polysaccharides), disintegrants (e.g., starch, cellulose, crosslinked polyvinyl pyrrolidone, sodium starch glycolate, sodium carboxymethyl-cellulosemethycellulose), fillers (e.g., cellulose, gelatin, calcium phosphate, vegetable fats and oils and sugars, such as lactose), diluents, flavors, colors, glidants (e.g., silicon dioxide, talc), lubricants (e.g., talc, silica, fats, stearin, magnesium strearate, stearic acid), preservatives (e.g., antioxidants such as vitamins A, E, C, selenium, systein, methionine, citric acids, sodium citrate, methyl paraben, propyl paraben), sorbents, sweeteners (e.g., syrup). In a particular non-limiting embodiment, the excipient comprises HEC (hydroxyethylcellulose), which is a nonionic, water-soluble polymer that can thicken, suspend, bind, emulsify, form films, stabilize, disperse, retain water, and provide protective colloid action.

Applicants also have discovered that mesenchymal stem cells from certain female donors expressed TSG-6 protein in general in increased amounts as compared to mesenchymal stem cells from male donors. Although Applicants do not intend to be limited to any theoretical reasoning, such discovery may be due, at least in part, to the periodic bursts or increases in female hormones during menstruation.

Thus, in accordance with another aspect of the present invention, there is provided a method of stimulating isolated mesenchymal stem cells to express increased amounts of tumor necrosis factor-α stimulating gene 6 (TSG-6) protein or a biologically active fragment, derivative, or analogue thereof. The method comprises contacting the isolated mesenchymal stem cells with at least one female hormone or derivative or analogue thereof in an amount of at least 50 nM, whereby the isolated mesenchymal stem cells express TSG-6 protein or a biologically active fragment, derivative, or analogue thereof in an amount greater than the amount of TSG-6 protein or a biologically active fragment, derivative, or analogue thereof expressed by the isolated mesenchymal stem cells prior to the contacting of the isolated mesenchymal stem cells with the at least one female hormone or derivative or analogue thereof in an amount of at least 50 nm.

Female hormones or derivatives or analogues thereof with which the isolated mesenchymal stem cells may be contacted include, but are not limited to, estradiol, estrogen, and progesterone. In a non-limiting embodiment, the at least one female hormone or derivatives or analogue thereof is estradiol.

In a non-limiting embodiment, the isolated mesenchymal stem cells are contacted with the at least one female hormone or derivative or analogue thereof in an amount of at least 100 nM. In another non-limiting embodiment, the isolated mesenchymal stem cells are contacted with the at least one female hormone or derivative or analogue thereof in an amount of at least 400 nM.

The isolated mesenchymal stem cells which are contacted with at least one female hormone or derivative or analogue thereof in an amount of at least 50 nM may be administered to an animal suffering from an inflammatory disease or disorder, such as those hereinabove described, in an amount effective to treat the inflammatory disease or disorder in the animal. In a non-limiting embodiment, the animal is a primate. In another non-limiting embodiment, the primate is a human.

Although Applicants have discovered that mesenchymal stem cells that produce mRNA encoding TSG-6 protein or a biologically active fragment, derivative, or analogue thereof in high amounts, such as an amount of at least a preselected amount are effective in treating diseases, disorders, and conditions associated with inflammation, Applicants also discovered that mesenchymal stem cells that produce low amounts of mRNA encoding TSG-6 protein or a biologically active fragment, derivative, or analogue thereof have increased osteogenic potential, i.e., have increased potential for differentiating into bone cells or bone tissues, and this may be useful in treating bone diseases, conditions, or disorders.

Thus, in accordance with another aspect of the present invention, there is provided a composition comprising isolated mesenchymal stem cells that produce mRNA encoding TSG-6 protein or a biologically active fragment, derivative, or analogue thereof in an amount that does not exceed a preselected amount, as measured by an assay. The assay comprises assaying the level of mRNA encoding TSG-6 protein or a biologically active fragment, derivative, or analogue thereof that is produced by a population of isolated mesenchymal stem cells, and determining, from the level of mRNA encoding TSG-6 protein or a biologically active fragment, derivative, or analogue thereof produced by the population of isolated mesenchymal stem cells, whether the population of isolated mesenchymal stem cells produce mRNA encoding TSG-6 protein or a biologically active fragment, derivative, or analogue thereof in an amount that does not exceed the preselected amount.

Although the scope of this aspect of the present invention is not intended to be limited to any theoretical reasoning, it is believed that, in general, mesenchymal stem cells that produce decreased amounts of mRNA encoding TSG-6 protein or a biologically active fragment, derivative, or analogue thereof also will express TSG-6 protein in decreased amounts. Therefore, mesenchymal stem cells that produce mRNA encoding TSG-6 protein or a biologically active fragment, derivative, or analogue thereof in amounts that do not exceed the preselected amount are more likely to express TSG-6 protein or a biologically active fragment, derivative, or analogue thereof in an amount such that the mesenchymal stem cells are useful particularly for treating bone diseases, conditions, and disorders, including bone injuries.

The mesenchymal stem cells may be obtained from an appropriate donor, and then isolated or purified by methods known in the art.

In a non-limiting embodiment, the isolated mesenchymal stem cells have been genetically engineered with a polynucleotide encoding TSG-6 protein or a biologically active fragment, derivative, or analogue thereof, as hereinabove described. Although mesenchymal stem cells would be genetically engineered with a polynucleotide encoding TSG-6 protein or a biologically active fragment, derivative, or analogue thereof in order to express increased amounts of TSG-6 protein or a biologically active fragment derivative, or analogue thereof, if the genetically engineered isolated mesenchymal stem cells produce mRNA encoding TSG-6 protein or a biologically active fragment, derivative, or analogue thereof in an amount that does not exceed the preselected amount and therefore are likely to express low amounts of TSG-6 protein or a biologically active fragment, derivative, or analogue thereof, such genetically engineered mesenchymal stem cells may be used to treat bone diseases, disorders, and conditions as described hereinbelow.

The isolated mesenchymal stem cells then are assayed for levels of mRNA encoding TSG-6 protein or a biologically active fragment, derivative, or analogue thereof in order to determine whether the isolated mesenchymal stem cells produce mRNA encoding TSG-6 protein or a biologically active fragment, derivative, or analogue thereof in an amount that does not exceed the preselected amount.

In another non-limiting embodiment, the population of isolated mesenchymal stem cells is assayed for levels of mRNA encoding TSG-6 protein or a biologically active fragment, derivative, or analogue thereof produced by the mesenchymal stem cells by conducting a RT-PCR assay.

In a non-limiting embodiment, the amount of mRNA encoding TSG-6 protein produced by a “standard” or “reference” population of mesenchymal stem cells is determined by a reverse transcription PCR assay. The amount of mRNA encoding TSG-6 protein produced by the “standard” or “reference” population of mesenchymal stem cells thus is the preselected amount. In a non-limiting embodiment, the “standard” or “reference” population is a population from a human donor known as Donor 7052 or a human donor known as Donor 7075. These cell populations have been found to produce similar amounts of mRNA encoding TSG-6 protein and are available from the institute for Regenerative Medicine, Texas A & M College of Medicine. The amount of mRNA encoding TSG-6 protein or a biologically active fragment, derivative, or analogue thereof produced by a test population of mesenchymal stem cells then is determined by the reverse transcription PCR assay. The test population of mesenchymal stem cells contains approximately the same number of mesenchymal stem cells as the “standard” or “reference” population. When the “standard” or “reference” population of mesenchymal stem cells is from Donor 7052 or Donor 7075 as hereinabove described, if the amount of mRNA encoding TSG-6 protein or a biologically active fragment, derivative, or analogue thereof produced by the test population is about the same or less than that produced by the “standard” or “reference” population, the mesenchymal stem cells from the test population are considered to be suitable especially for treating bone diseases and disorders and conditions, including bone injuries.

The isolated mesenchymal stem cells, which have been determined by an assay to produce mRNA encoding TSG-6 protein or a biologically active fragment, derivative, or analogue thereof in an amount that does not exceed a preselected amount may be administered in an amount effective to treat a bone disease, disorder, or condition in a vertebrate animal. In a non-limiting embodiment, the vertebrate animal is a primate, which includes human and non-human primates.

Although the scope of this aspect of the present invention is not to be limited to any theoretical reasoning, it is believed that mesenchymal stem cells that produce mRNA encoding TSG-6 protein or a biologically active fragment, derivative, or analogue thereof in an amount that does not exceed a preselected amount may be more likely to differentiate in vivo into bone producing cells, i.e., osteoblasts. Thus, such mesenchymal stem cells may be better able to repair diseased or injured bone.

Bone diseases, disorders, and conditions which may be treated by the isolated mesenchymal stem cells selected in accordance with this aspect of the present invention include, but are not limited to, osteoarthritis, osteoporosis, osteosarcoma, jaw bone damage, or maxillary bone damage caused by periodontal disease, spinal column diseases and injuries, and bone fractures.

The isolated mesenchymal stem cells, which produce mRNA encoding TSG-6 protein or a biologically active fragment, derivative, or analogue thereof in an amount that does not exceed a preselected amount may be administered systemically, such as, for example, by intravenous, intraarterial, intraperoneal, intramuscular, or subcutaneous administration. Alternatively, the mesenchymal stem cells may be administered directly to the bone of said patient.

The isolated mesenchymal stem cells, which produce mRNA encoding TSG-6 protein or a biologically active fragment, derivative, or analogue thereof in an amount that does not exceed a preselected amount are administered in conjunction with an acceptable pharmaceutical carrier such as those hereinabove described.

In accordance with another aspect of the present invention, there is provided a kit for determining the presence and/or amount of an RNA sequence encoding TSG-6 protein or a biologically active fragment, derivative, or analogue thereof in mesenchymal stem cells. The kit comprises a preparation of mesenchymal stem cells that produce a predetermined amount of an RNA sequence encoding TSG-6 protein or a biologically active fragment, derivative, or analogue thereof. The kit also comprises at least two identical culture media for culturing and expanding mesenchymal stem cells and instructions for culturing and expanding the mesenchymal stem cells.

Also included in the kit are at least two identical sets of reagents for extracting RNA from mesenchymal stem cells and instructions for extracting RNA from the mesenchymal stem cells. The kit further comprises at least three microplates suitable for conducting reverse transcription PCR, or RT-PCR, of RNA.

The kit also contains a predetermined amount of an RNA sequence encoding TSG-6 protein or a biologically active fragment, derivative, or analogue thereof. The predetermined amount of the RNA sequence encoding TSG-6 protein or a biologically active fragment, derivative, or analogue thereof was extracted previously from the mesenchymal stem cells hereinabove described. The predetermined amount of the RNA sequence, in a non-limiting embodiment, is pre-loaded onto at least one of the at least three microplates suitable for conducting reverse transcription PCR of the RNA.

The kit also includes a 3′ DNA primer and a 5′ DNA primer corresponding to the RNA sequence encoding TSG-6 protein or a biologically active fragment, derivative, or analogue thereof of which the presence and/or amount thereof is to be determined.

The kit further includes at least two identical sets of reagents for conducting reverse transcription PCR.

Furthermore, the kit includes instructions for conducting reverse transcription PCR of RNA, and instructions for assaying for the presence and/or amount of the RNA sequence encoding TSG-6 protein or a biologically active fragment, derivative, or analogue thereof.

RNA sequences encoding TSG-6 protein or a biologically active fragment, derivative, or analogue thereof which may be detected by the kit of the present invention include, but are not limited to, messenger RNA, or mRNA, transfer RNA, or tRNA, and ribosomal RNA, or rRNA.

The mesenchymal stem cells that produce a predetermined amount of the RNA sequence encoding TSG-6 protein or a biologically active fragment, derivative, or analogue thereof can be obtained from any animal, including human and non-human animals, and any tissue or other cellular source in which mesenchymal stem cells are present. In a non-limiting embodiment, the mesenchymal stem cells are obtained from a human. In another non-limiting embodiment, the mesenchymal stem cells are obtained from human bone marrow. In another non-limiting embodiment, the mesenchymal stem cells are produced from induced pluripotent stem cells.

In another non-limiting embodiment, the mesenchymal stem cells have been genetically engineered with a polynucleotide encoding TSG-6 protein or a biologically active fragment, derivative, or analogue thereof.

In a non-limiting embodiment, the mesenchymal stem cells contained in the kit are supplied as a frozen vial to be stored under liquid nitrogen. Each vial contains 0.75 to 1.0 million cells in 1 ml of α-minimum essential medium (α-MEM) (Gibco), 5% dimethylsulfoxide (DMSO), and 20% fetal bovine serum (Atlanta Biologicals).

The culture media used for culturing and expanding the mesenchymal stem cells may be any culture media known to those skilled in the art for culturing and expanding mesenchymal stem cells. In a non-limiting embodiment, the kit contains at least two identical samples of culture media in an amount of about 100 ml.

In a non-limiting embodiment, the at least two identical samples of culture media contain complete culture medium (CCM) consisting of a-minimum essential medium (α-MEM) supplemented with 17% fetal bovine serum (FBS, Atlanta Biologicals), 100 units/ml penicillum (Gibco), 100 μg/ml streptomycin (Gibco), and 2 mM L-glutamine (Gibco).

The instructions for culturing and expanding the mesenchymal stem cells in general direct one to culture and expand the mesenchymal stem cells under conditions and for a period of time sufficient to provide an amount of mesenchymal stem cells from which a sufficient amount of RNA can be extracted from the cells. In a non-limiting embodiment, the instructions direct one to culture the mesenchymal stem cells in the medium for a total period of time of from about 6 days to about 8 days.

In a non-limiting embodiment, the instructions instruct one skilled in the art to thaw the frozen vials of the mesenchymal stem cells at 37° C., and then suspend the mesenchymal stem cells in 100 ml of the complete culture medium (CCM). The instructions then instruct one to plate the cells on a 152 cm² culture dish (Corning), and then to wash the cells with phosphate buffered saline, and to harvest adjacent cells by exposure to 0.25% trypsin and 1mM ethylenediaminetetracetic acid (EDTA) (Gibco) for 2 to 7 minutes. The instructions then instruct one to plate the cells in 100 ml CCM at 200 cells/cm², replace the medium after 3 days, and lift the cells with 0.25% trypsin and 1 mM EDTA after 5 days.

The RNA may be extracted from the mesenchymal stem cells with any reagents for extracting RNA from cells that are known to those skilled in the art. In a non-limiting embodiment, the kit includes a “sub kit” that contains the reagents and other materials for extracting RNA from cells. An example of such a “sub-kit” is the RNeasy Mini Kit, sold by Qiagen Inc. Such “sub-kit” also contains appropriate instructions for extracting RNA from cells. In another non-limiting embodiment, the “sub-kit” is the High Pure RNA Isolation Kit (catalog no. 11828665001, Roche).

The microplates which are contained in the kit may be any microplates known to those skilled in the art to be suitable for conducting reverse transcriptase PCR of RNA.

The 3′ and 5′ DNA primers contained in the kit may any 3′ and 5′ DNA primers that are appropriate for reverse transcription PCR. The sequences of such primers are determined in part by the RNA sequences encoding TSG-6 protein or a biologically active fragment, derivative, or analogue thereof that one wishes to detect.

The reagents for conducting reverse transcription PCR may be any of those known to one skilled in the art, including reverse transcriptase, dATP, dGTP, dCTP, and dTTP.

In a non-limiting embodiment, the microtiter plates, 3′ and 5′ primers, and reagents are supplied as the Custom Profiler RT 2 PCR Array (www.sasciences.com), which includes the microtiter plates preloaded with the appropriate 3′ and 5′ DNA primers, and the reagents to develop the reverse transcription PCR reactions.

The reverse transcription PCR is conducted in accordance with the instructions provided in the kit. Such instructions will direct one to conduct the reverse transcription PCR according to any of a variety of procedures known to those skilled in the art. Examples of such procedures may be contained in the Custom Profiler RT2 PCR Array, or may be those described in Wu, et al., Methods in Gene Biotechnology, CRC Press (1997), pgs. 16-21.

The kit contains means for determining the presence and/or amount of the RNA sequence encoding TSG-6 protein or a biologically active fragment, derivative, or analogue thereof, plus instructions for using such means. Such means may be any of those known to those skilled in the art. Examples of such means includes, but are not limited to Sequence Detection Software V2.3 (Life Technologies) and the comparative CT method using RQ manager V1.2 (Life Technologies).

The kit of the present invention is applicable particularly to determining the presence and/or amount of an RNA sequence encoding TSG-6 protein or a biologically active fragment, derivative, or analogue thereof in a test population of mesenchymal stem cells from any source and obtained by any procedure known to those skilled in the art Parallel experiments are conducted in which the test population of mesenchymal stem cells and the population of mesenchymal stem cells producing a predetermined amount of the RNA sequence encoding TSG-6 protein or a biologically active fragment, derivative, or analogue thereof are cultured and expanded. RNA then is extracted from both populations of cells, and reverse transcription PCR is conducted on both of the extracted RNAs. Reverse transcription PCR also is conducted on the predetermined amount of RNA sequence encoding TSG-6 protein or a biologically active fragment, derivative, or analogue thereof extracted previously from the mesenchymal stem cells producing the predetermined amount of RNA sequence encoding TSG-6 protein or a biologically active fragment, derivative, or analogue thereof in order to verify the accuracy of the experiments. Then, the presence and/or amount of RNA sequence encoding TSG-6 protein or a biologically active fragment, derivative, or analogue thereof produced by the test population of mesenchymal stem cells is compared with the amount of RNA sequence encoding TSG-6 protein or a biologically active fragment, derivative, or analogue thereof produced by the mesenchymal stem cells that produce a predetermined amount of such RNA sequence encoding TSG-6 protein or a biologically active fragment, derivative, or analogue thereof. Through such a comparison, one can determine whether the test population of mesenchymal stem cells is suitable for a variety of therapeutic applications including but not limited to, the treatment of inflammatory diseases or disorders, or bone diseases, disorders, and conditions hereinabove described.

### BRIEF DESCRIPTION OF THE DRAWINGS

The invention now will be described with respect to the drawings, wherein: FIG. 1. Correlation between potential biomarkers and effectiveness in reducing MPO levels in the injured cornea. (A) Quantification of corneal opacity and infiltrating neutrophils as measured by the myeloperoxidase concentration on day 3 after injury. (B-C) Mice received IV injection of hMSCs (1×10⁶) from 11 different donors or HBSS. (B) Representative corneal photographs on day 7 following injury. (C) Quantification of infiltrating neutrophils as measured by the myeloperoxidase (MPO) concentration in the cornea on day 1 after injury. (n=3 to 5 for 11 donor hMSCs; n=6 for HBSS, *, P<0.05; **, P<0.01; ***, P<0.001; N.S., no significant difference; one-way ANOVA with Dunnett's Multiple Comparison Test). (D) Correlations between efficacy of hMSCs in reducing MPO levels in the cornea model and standard in vitro assays for MSCs, age, gender, height, and weight of donors of marrow aspirates. (E) hMSC morphology from donors 235, 269, 6015, 7052, 7074, and 7075 as shown in representative hMSC photographs at day 4 after being plated at 200 cells/cm² in 6 wells. (F) Correlations between efficacy of hMSCs in reducing MPO levels in the cornea model and expression by RT-PCR of genes previously linked to the therapeutic benefits of hMSCs. Upper and lower panels indicate values obtained with or without stimulation of the cells with TNF-α. IDO1 was not detected in all preparations that were not stimulated. (G) Comparisons of values obtained with hMSCs from male and female donors for osteogenic potential (15 donors), and TSG-6 expression by RT-PCR with (20 donors) and without (23 donors) TNF-α stimulation.

FIG. 2. Effects of estradiol on hMSCs from male donor. (A) hMSCs (male donor 7052) were incubated with different concentrations of estradiol for 24 hrs. and real time RT-PCR was performed for TSG-6 expression. (B) hMSCs (donor 7052, 1,000 cells/cm²) were treated with 100 nM estradiol for 48 hrs., the increased levels of TSG-6 mRNA were observed after the cells were incubated without estradiol for an additional 2 days. (C) Representative hMSC photographs after estradiol treatment for 4 days. (D-E) hMSCs (donor 7052, 1000 cells/cm²) were treated with different concentrations of estradiol every 2 days for 4 days and then cells were changed with osteogenic medium every 2-3 days for 2 weeks. Representative photographs of Alizarin Red staining (D) and quantification (E) of Alizarin Red staining. (n=3; ***, P<0.001; one-way ANOVA).

FIG. 3. The TSG-6^{hi} hMSCs are more effective in suppressing sterile inflammation in three in vivo models. (A) Expression levels in a series of donors of bone marrow aspirates/hMSCs of TSG-6 by RT-PCR with and without TNF-α stimulation. Values are relative to expression level in donor 7075 that was set as 1.0. To simplify comparisons of the data from experiments in vivo, three preparations with the highest levels were designated as TSG-6^{hi} and three with the lowest levels were designated as TSG-6^{low}. Each of the six preparations was tested separately. (B) Quantification of corneal opacity on day 7 using clinical grading system on a 0-4 scale. The values are data from 3 to 5 mice. Each mouse was treated with MSCs from the same donor of which 3 were TSG-6^{hi} and 3 were TSG-6^{low}. (***,P<0.001; N.S, no significant difference; one-way ANOVA) (C) Quantification of infiltrating neutrophils as measured by the myeloperoxidase (MPO) concentration in the cornea on day 1 following injury. (n=4 or 5 mice for each of 3 different MSC donors and n=12 for HBSS control, **, P<0.01; ***, P<0.001; one-way ANOVA) (D) Assays of the efficacy of the hMSCs in the model for zymosan-induced peritonitis. Values are from ELISA assays for mouse TNF-α, CXCL1, and CXCL2 in peritoneal lavage of mice. The peritoneal lavage was collected at 4 hrs. after zymosan injection (IP) followed by hMSCs injection (IP, 1.5×10⁶). (n=3 for each donor of which 3 were TSG-6^{hi} and 3 were TSG-6^{low} as shown in FIG. 3A and n=7 for HBSS control; *, P<0.05; **,P<0.01; ***,P<0.001; N.S, no significant difference; one-way ANOVA). (E) Survival proportions after bleomycin injury of lung in mice followed by treatment with hMSC (IV, 2.5×10⁵ cells; hMSCs (n=5 or 6) from the same donor of which 3 were TSG-6^{hi} and 3 were TSG-6^{low} as shown in FIG. 3A) or HBSS control (n=15) by Log-rank (Mantel-Cox) test. (F-H) Survival proportions, relative weight changes, and oxygen saturation levels after bleomycin injury followed by treatment with hMSCs. (F-G) The survival proportions of mice that received hMSCs (n=5 or 6) from the same donor of which 3 were TSG-6^{hi} and 3 were TSG-6^{low} as shown in FIG. 3A or HBSS control (n=15). (H) Relative weight changes prior to death or end point were expressed as a percentage of pre-injury weight (by one-way ANOVA).

FIG. 4. Negative correlation between osteogenic differentiation potential and TSG-6 expression. (A) Correlation between osteogenic differentiation potential and the levels of mRNA for TSG-6 in hMSCs with TNF-α stimulation (5 ng/ml for 16 hrs.). (B) Correlation between the levels of mRNA for TSG-6 and TNFRSFIA in hMSCs. (C) Nuclear extracts from TSG-6^{hi} and TSG-6^{low} hMSCs were assayed for NF-κB DNA binding activity by EMSA. The specific DNA-binding activity of NF-κB complex is indicated by an arrow. (D) Real time RT-PCR for the levels of TSG-6 in hMSCs (TSG-6^{hi} donor 6015) after 24 hr. treatment of SN50, NF-κB inhibitor. (E) Representative photo of Alizarin Red staining on SN50 pretreated TSG-6^{hi} hMSCs prior to osteogenic differentiation. (F) Quantification of Alizarin Red staining of FIG. 3E. (n=3; ***, P<0.001; one-way ANOVA). (G) Real time RT-PCR for the levels of TSG-6 in hMSCs (TSG-6^{low} donor 7052) transfected with control vector (7052^{Δcont}) or TSG-6 (7052^{ΔTSG-6}) after 24 hrs. (H) Representative photo of Alizarin Red staining on 7052^{Δcont} and 7052^{ΔTSG-6} after osteogenic differentiation. (I) Photographs of mice that received IV injections of 1×10⁶ cells of 7052^{Δcont} or 7052^{ΔTSG-6} after corneal injury. (J) Quantification of corneal opacity on day 3 using clinical grading system on a 0-4 scale. (n=7 or 8 for each groups, **, P<0.01; ***,P<0.001; N.S., no significant difference; one-way ANOVA) (K) Quantification of infiltrating neutrophils as measured by the myeloperoxidase (MPO) concentration in the cornea on day 1 after injury. (n=6 to 8 for each groups, *, P<0.05; **, P<0.01; ***, P<0.001; one-way ANOVA). (J) Representative corneal photographs on day 3 after injury.

### EXAMPLE

The invention now will be described with respect to the following example. It is to be understood, however, that the scope of the present invention is not intended to be limited thereby.

### Materials and Methods

### Cell Preparations

hMSCs were prepared as described previously (Sekiya, 2002; Roddy, 2011; Choi et al., 2011; Lee et al., 2009). The aspirates were obtained over several years from normal volunteers who responded to local postings in an academic setting and who were screened beforehand with blood assays for infectious agents. Further information on the bone marrow samples and the donors is shown in Table 1 below.

TABLE 1
Information for hMSC donors.

| Bone | | | | | | |
|---|---|---|---|---|---|---|
| Marrow | | | | | | Left; Right |
| Sample | Sample | | | Weight | Height | Aspirate |
| No | Date | Sex | Age | (lbs) | (Inches) | Volume |
| 235 | Jan. 6, 2004 | F | 24.10 | 120 | 63 | 3 |
| 260 | Apr. 6, 2004 | F | 30.30 | 115 | 64 | 2 |
| 269 | May 11, 2004 | F | 31.50 | 112 | 64 | 3 |
| 5046 | Nov. 30, 2004 | F | 40.80 | 112 | 65 | 3 |
| 5062 | Jan. 4, 2005 | F | 21.00 | 156 | 65 | 3; 4 |
| 6015 | Mar. 22, 2005 | F | 20.50 | 128 | 63 | 2.5 |
| 6091 | Jun. 28, 2005 | F | 22.10 | 156 | 66 | 3 |
| 7012 | Jul. 19, 2006 | F | 26.70 | 172 | 64 | 2; 2 |
| 7013 | Jul. 19, 2006 | F | 33.00 | 135 | 70 | 2 |
| 7015 | Aug. 9, 2006 | F | 29.70 | 125 | 65 | 2 |
| 7027 | Jan. 31, 2007 | M | 47.00 | 175 | 75 | 2; 2 |
| 7043 | Jul. 5, 2007 | M | 28.75 | 147 | 65 | 3 |
| 7049 | Sep. 13, 2007 | F | 25.70 | 145 | 62 | 2; 2 |
| 7052 | Oct. 30, 2007 | M | 20.30 | 126 | 69 | 2 |
| 7055 | Nov. 6, 2007 | F | 59.33 | 165 | 65 | 3 |
| 7064 | Jan. 2, 2008 | M | 24.20 | 178 | 72 | 2; 2 |
| 7068 | Mar. 4, 2008 | M | 37.20 | 230 | 72 | 1 |
| 7073 | Mar. 26, 2008 | M | 21.60 | 175 | 73 | 2 |
| 7074 | Apr. 1, 2008 | M | 26.30 | 180 | 68 | 2 |
| 7075 | Apr. 15, 2008 | M | 24.20 | 161 | 72 | 2; 2 |
| 8006 | Jan. 25, 2012 | M | 23.00 | 200 | 72 | 2; 4 |

In brief, mononuclear cells were isolated by ficoll gradient separation of bone marrow from the iliac crest of normal volunteers, incubated in complete culture medium (CCM) [α-MEM (Life technologies, Carlsbad, Calif.) containing 17% (v/v) FBS (Atlanta Biologicals, Lawrenceville, Ga.), 2 mM L-glutamine and 1% (v/v) penicillin-streptomycin (Life Technologies)] at high density to obtain adherent cells (P0 cells), replated at low density (60 to 100 cells/cm²), incubated to about 70% confluency (cell density about 10,000 cells/cm² at harvest), and frozen (P1 cells, 1×10⁶ cells/vial). Frozen vials of P1 cells were thawed and incubated at high density to obtain adherent viable cells, replated at low density (200 cells/cm²), and incubated to about 70% confluency (cell density about 10,000 cells/cm² at harvest) to obtain P2 hMSCs that were used for the experiments.

To activate the cells to express TSG-6, P2 hMSCs were incubated with 5 ng/mL of TNF-α (R&D Systems, Minneapolis, Minn.) in α-MEM containing 2% FBS for 16 hrs. (Sekiya, 2002; Lee et al., 2009). Similar results were obtained with 2 or more vials of P1 MSCs from the same master bank prepared from the same donor. The FBS used for the experiments were selected by screening 4 to 5 lots for rapid growth of MSCs. Different lots standardized to provide about the same propagation rate of MSCs were used to prepare P0 MSCs, but the same lot was used to expand P1 to P2 MSCs for the experiments here.

RNA Extraction from Cultured Cells and Real Time RT-PCR Analysis

Total RNA from monolayer cells was extracted (RNeasy Mini Kit; Qiagen, Germantown, Md.) and about 0.1-1 ug of total RNA per sample was used to synthesize double-stranded cDNA by reverse transcription (SuperScript III; Life Technologies). Real-time RT-PCR was performed in triplicate for hGapdh, TSG-6 (TNFAIP6), HMOX1, COX2, IL1Ra, TGF-β1, IDO1, and TNFRSF1A, using Taqman Gene Expression Assays (Life Technologies). Real-time amplification was performed with TaqMan Universal PCR Master Mix (Life Technologies) and analyzed on 7900HT fast real-time PCR system (Life Technologies). For assays, reactions were incubated at 50° C. for 2 min, 95° C. for 10 min, and then 40 cycles at 95° C. for 15 seconds followed by 60° C. for 1 min. Data were analyzed with Sequence Detection Software V2.3 (Life Technologies) and relative quantities (RQs) were calculated with comparative CT method using RQ Manager V1.2 (Life Technologies).

### Animals

The experimental protocols were approved by the Institutional Animal Care and Use Committee of Texas A&M Health Science Center. Six-to seven week-old male BALB/c mice (BALB/cAnNCrI; Charles River Laboratories International) were used in all experiments.

### Animal Model of Injury and Treatment

Chemical burned corneal injury was produced as described previously (Oh et al., 2010) Mice were anesthetized by isoflurane inhalation. To create the chemical burn, 100% ethanol (Sigma-Aldrich, St. Louis, Mo.) was applied to the whole cornea including the limbus for 30 seconds followed by rinsing with 1 mL of Phosphate-Buffered Saline (PBS, Life Technologies). Then, the epithelium over the whole corneal and limbal region was mechanically scraped using a surgical blade. Upon completion of the procedure, the eyelids of the mice were closed with one 8-0 silk suture at the lateral third of the lid margin. Immediately following injury, mice received an intravenous (IV) injection of hMSCs (1×10⁶) in 0.1 mL Hank's Balanced Salt Solution (HBSS, Life Technologies).

### Ocular Surface Evaluation

After injury and treatment, the mouse corneas were examined for corneal opacity and photographed at 3 or 7 days. Corneal opacity was assessed and graded as described previously from the photographs by an ophthalmologist who was not aware of the treatment of the mice (Oh et al., 2010).

Protein Extraction from Cornea

For protein extraction from cornea, corneas were lysed in 150 μL of tissue extraction reagent containing protease inhibitors (Life Technologies). The samples were sonicated on ice and centrifuged at 15,000×g at 4° C. for 15 min. The supernatant was used for MPO ELISA assays.

### Mouse Model of Peritonitis and Measurements of Inflammation

To induce inflammation in male BALB/c mice, 1 ml of zymosan solution (1 mg/mL) was administered by IP, followed by IP injection of 1.5×10⁶ each donor derived hMSCs 15 min later (Roddy, et al; 2011; Choi et al., 2011). After 4 hrs., inflammatory exudates were collected by peritoneal lavage and the cell-free supernatant was used to measure levels of the proinflammatory molecules (mTNFα, mCXCL1, and mCXCL2/MIP-2) by ELISA assays.

Mouse Model of Lung Injury Induced with Bleomycin

Lung injury was induced in female C57BL/6J mice anesthetized with isofluorane by administration of bleomycin sulfate (Sigma-Aldrich Corp.) at 2.25 U/kg of body weight in 0.9% sodium chloride via intubation technique (Foskett, et al., 2014). Sham animals were given 0.9% sodium chloride alone. IV administration of each donor-derived hMSC (2.5×10⁵ cells in 150 μl) was performed on days 1 and 4 post-injury. A portable mouse pulse oximeter (STARR Life Sciences Corp.) was used to monitor arterial blood oxygen saturation (SpO₂) in free-roaming non-anesthetized mice. Weight and SpO₂ measurements were recorded every other day for the entire duration of the 21-day survival study.

### ELISA Assays

Mouse MPO (mouse MPO ELISA kit; HyCult Biotech, Plymouth Meeting, Pa.), TNF-α, CXCL1, and CXCL2 (R&D Systems) were detected with commercially available ELISA kits following procedures described by the manufacturers.

### TSG-6 Overexpression

Total RNA was isolated from hMSCs stimulated with 10 ng/mL of TNF-α in α-MEM containing 2% FBS overnight (Sekiya, 2002; Lee et al., 2009). About 1 μg of total RNA was used to produce the first strand cDNA pool by Reverse Transcriptase (Superscript II/oligo dT12-18, Life Technologies). cDNAs encoding hTSG-6 (GenBank accession number: NM _007115) were amplified by PCR using the following primers: 5′-CGGGGTACCATGATCATCTTAATTTACTT-3′ (sense for hTSG-6), and 5′-GGTGATCAGTGGCTAAATCTTCCA-3′ (anti-sense for hTSG-6-WT). The PCR products were sub-cloned into the BamHI and EcoRI sites in multi-cloning sites of a pEF4-Myc/His plasmid (Life Technologies) and the plasmids were amplified in E. coli DH5α cells (Life Technologies). The TSG-6 or control plasmid (0.1 μg/well in 6 wells) was transfected in hMSC with lipofectamine 2000 (Life Technologies) according to the manufacturer's protocol. Twenty-four hours after transfection, the cells were harvested for assays.

### Differentiation Assay

hMSCs were plated at 10,000 cells/cm² in a six well plate. To induce adipogenesis, hMSCs were cultured in CCM supplemented with 500 nM dexamethasone (Sigma-Aldrich), 500 nM isobutylmethyixanthine (Sigma-Aldrich), and 50 μM indomethacin (Sigma-Aldrich) for 14 days with medium changes every 2-3 days. To induce osteogenesis, hMSCs were cultured in CCM supplemented with 10 nM dexamethasone, 10 mM β-glycerolphosphate (Sigma-Aldrich), and 50 βM ascorbate-2-phosphate (Sigma-Aldrich) for 18 days with medium changes every 2-3 days. For quantitative assays of adipogenic differentiation, the monolayer cells were fixed in 10% formalin for 10 min., washed three times with PBS and stained with fresh Oil Red-O solution in 60% (v/v) isopropyl alcohol in PBS for 20 min. The samples were washed extensively with PBS to remove unbound dye, and then 1 mL of isopropyl alcohol was added to the stained culture dish. After 5 min., the absorbance of the extract was assayed by a spectrophotometer (Fluostar Optima; BMG Labtechnologies, Offenburg, Germany) at 485 nm. For quantitative assay osteogenic differentiation, the cellular aggregates were washed in PBS and fixed in formalin for 30 min. The cells were stained with 40 mM Alizarin Red S for 30 min and washed with distilled water. The stained cells were transferred to a 2-ml screw-top microcentrifuge tube and incubated at 85° C. for 15 min in 1 ml of 10% (v/v) acetic acid (Chen, et al., 2010; Gregory et al., 2004). The extract was cooled on ice and centrifuged at 21,000×g for 5 min. About 0.5 ml of the supernatant was transferred to a fresh tube containing 0.2 ml of 10% (v/v) ammonium hydroxide. The red solution was transferred to a 96-well plate and read at 485 nm on a spectrophotometer.

### Nuclear Extraction and NF-κB Electrophoretic Mobility Shift Assay (EMSA)

Cells were harvested at density of 10,000 cells/cm² and nuclear fraction was extracted using a Nuclear Extraction Kit (Signosis, Santa Clara, Calif.) and EMSA for the detection of nuclear NE-KB was performed using EMSA Kit (Signosis) according to the manufacturer's instructions.

### Inhibition of NF-κB Signaling

hMSCs were plated at 10,000/well in CCM in 6-well plates. To inhibit NF-κB signaling, cells were treated with the NF-κB inhibitor, SN50 (EMD Millipore, Billerica, Mass.) in CCM every 2 days for 4 days. Then, the medium was changed to osteogenic differentiation media. For RT-PCR, cells were treated with SN50 (50-200 ng/ml) for 24 hours in CCM and harvested for RNA extraction.

### Statistical Analyses

Comparisons between two groups were made with the use of unpaired and two tailed Student's t tests. Comparison of more than two groups were evaluated by ANOVA. Survival of mice between groups was compared using log-rank (Mantel-Cox) Test. P<0.05 was considered significant.

### Results

We demonstrated previously that intravenous (IV) infusion of bone marrow hMSCs prevented the development of opacity following the chemical injury to the cornea by suppressing sterile inflammation (Oh, 2010) and that the efficacy of the MSCs was proportional to the decrease in MPO in the cornea (FIG. 1A) (Oh, 2010). We used the same model recently and found that large differences in the efficacy of 11 different preparations of hMSCs isolated and expanded from different donors of bone marrow (FIGS. 1B and 1C). Some (Donors 235, 269, and 6015) were highly effective, and others (Donors 7052, 7074 and 7075) provided little protection (FIG. 1B).

In order to identify a biomarker that predicts efficacy of hMSCs in the model, we assayed the same cells with conventional in vitro assays used to characterize MSCs (Sekiya, 2002, Digirolamo, 1999). Surprisingly, the values obtained in the assays showed no correlation with hMSC efficacy in vivo. In fact, there was a negative correlation with the potential for osteogenic differentiation in vitro (FIG. 1D). Also, there was no correlation with adipogenic potential, rate of proliferation and colony forming units-fibroblastoid (CFU-s). In addition, there was no significant correlation with age in assays on 11 donors who ranged from 20 to 70 years of age (FIG. 1D). Similarly, there was no apparent relationship to the spindle-shaped morphology of cells (FIG. 1E) that has been used to identify early progenitors in the cultures, (Owen, 1988; Colter, 2001) and no difference in expression of surface markers between the effective and ineffective hMSCs (Table 2).

TABLE 2
Expression of cell surface markers (SCM) in hMSCs.

| | Donor No. | | | | | | | | | | | |
|---|---|---|---|---|---|---|---|---|---|---|---|---|
| | 7075 | | 7052 | | 7074 | | 6015 | | 235 | | 269 | |
| | % | X-mean | % | X-mean | % | X-mean | % | X-mean | % of | X-mean | % | X-mean |
| | of (+) | of (+) | of (+) | of (+) | of (+) | of (+) | of (+) | of (+) | (+) | of (+) | of (+) | of (+) |
| SCM | cells | cells | cells | cells | cells | cells | cells | cells | cells | cells | cells | cells |
| CD73 | 99.9 | 166 | 99.9 | 143 | 100 | 149 | 100 | 145 | 99.9 | 158 | 99.9 | 113 |
| CD90 | 100 | 2190 | 91.9 | 2070 | 100 | 1950 | 91.9 | 2310 | 100 | 1500 | 100 | 2050 |
| CD105 | 100 | 311 | 99.9 | 270 | 99.9 | 288 | 99.9 | 289 | 100 | 332 | 99.9 | 299 |
| CD146 | 99.9 | 206 | 99.9 | 238 | 100 | 283 | 100 | 217 | 100 | 221 | 99.9 | 588 |
| CD147 | 100 | 285 | 99.9 | 235 | 100 | 253 | 99.9 | 302 | 100 | 276 | 99.9 | 308 |
| CD29 | 100 | 303 | 100 | 256 | 100 | 222 | 100 | 285 | 100 | 212 | 100 | 242 |
| CD166 | 100 | 489 | 91.9 | 536 | 100 | 484 | 100 | 558 | 100 | 461 | 100 | 450 |
| HLA | 99.9 | 126 | 99.9 | 78.4 | 99.9 | 114 | 100 | 160 | 99.9 | 112 | 99.8 | 113 |
| a, b, c | | | | | | | | | | | | |
| HLA II | 0.03 | 12.1 | 0.03 | 10.6 | 0.04 | 22.5 | 0.04 | 14 | 0.04 | 12.4 | 0.07 | 26.7 |

Simple RT-PCR assays for therapeutic genes that have been suggested as responsible for anti-inflammation/immune suppressive effects of hMSCs (Lee, 2009; Roddy, 2011; Kota, 2013; Choi, 2011; Ortiz, 2007; Nemeth, 2009; English, 2013; Meisel, 2004; Lee, 2011), however, predicted in vivo efficacy of hMSCs (FIG. 1F). The most significant correlation was with values for TNFα-stimulated gene 6 (TSG-6) mRNA in the hMSCs. The correlation essentially was the same if based on assays of hMSCs that were isolated freshly from culture and administered directly to the mice, or assays of the same cells after expression of TSG-6 was increased (Lee, 2009) by incubation with TNF-α for 16 hours. There is a slight positive correlation with the levels of heme oxygenase 1 (HMOX1) in hMSCs that were isolated freshly from culture, but not in hMSCs that were incubated with TNF-α. In addition, there were no significant correlations with the levels of mRNA for cyclooxygenase 2 (COX2), a key enzyme of synthesis of PGE2, IL-1 receptor antagonist (IL-1Ra), transforming growth factor-β1 (TGF-β1), or indoleamine 2-3 dioxygenase 1 (IDO1).

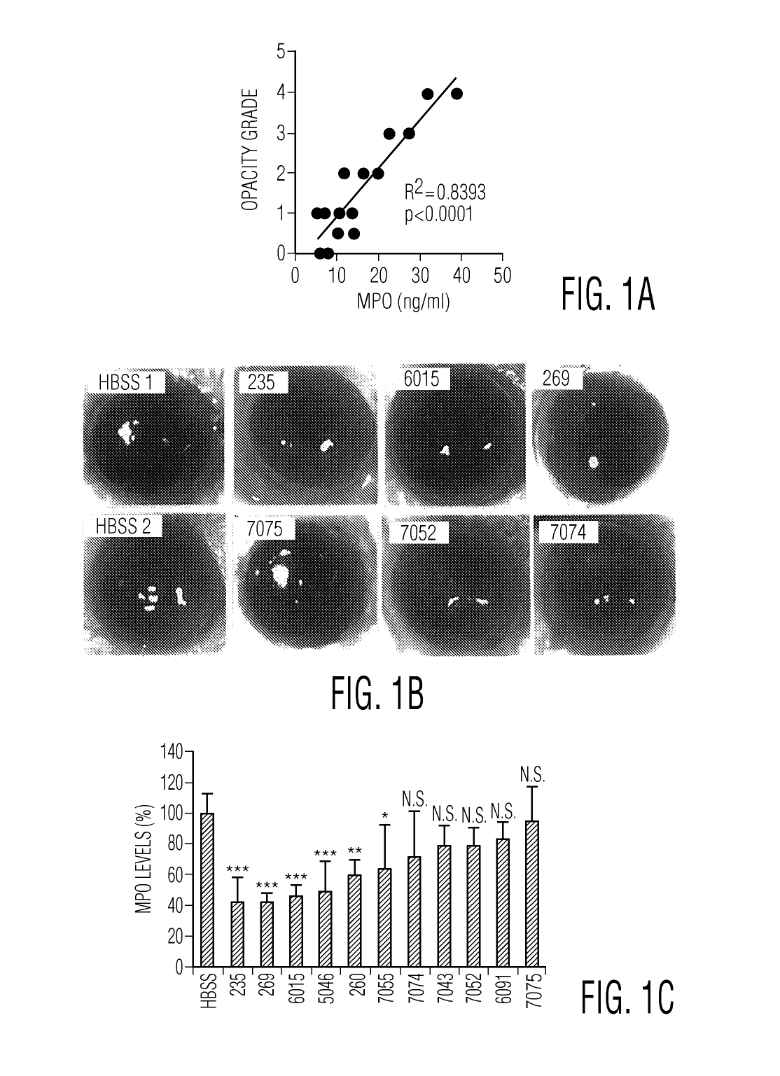

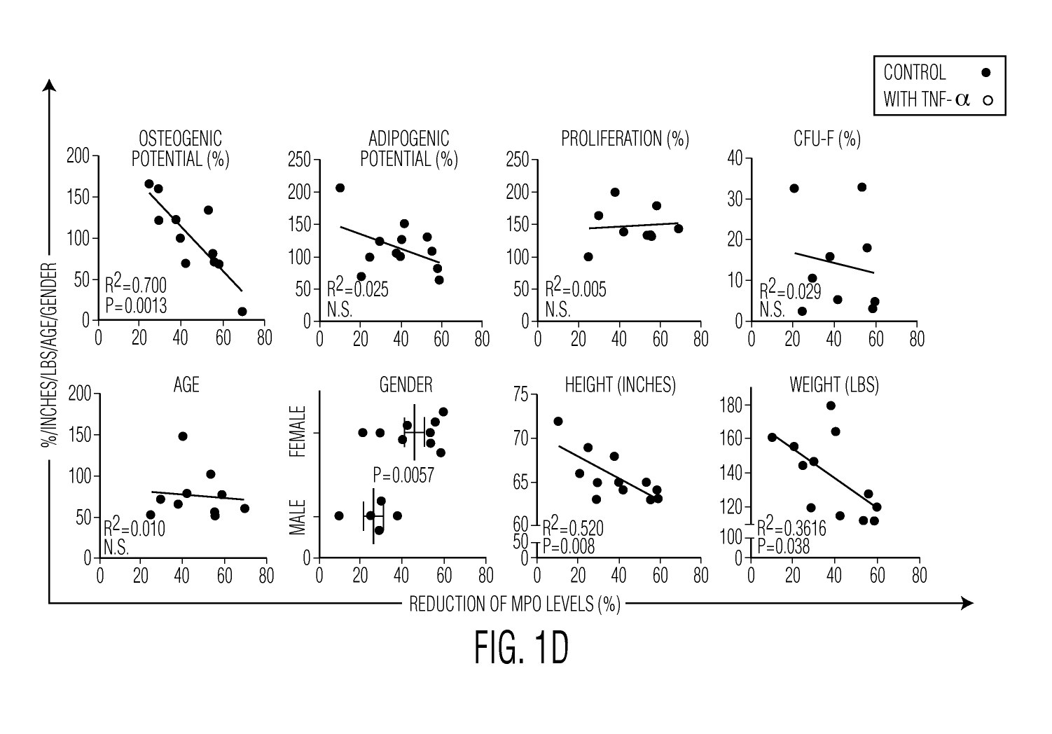

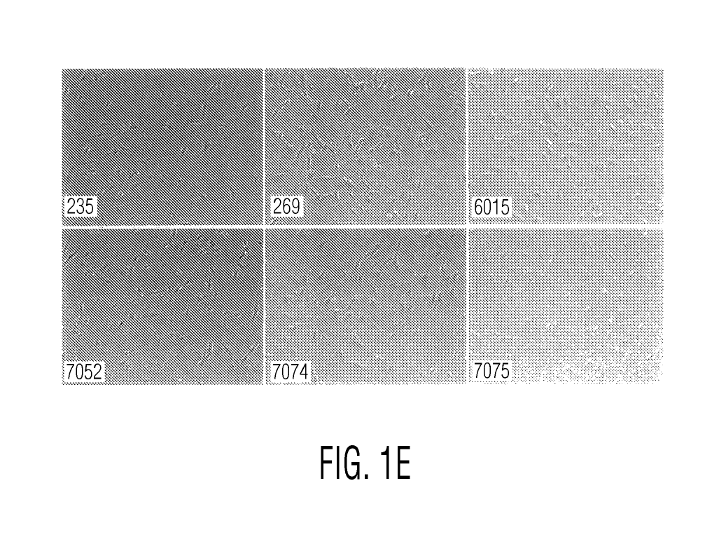

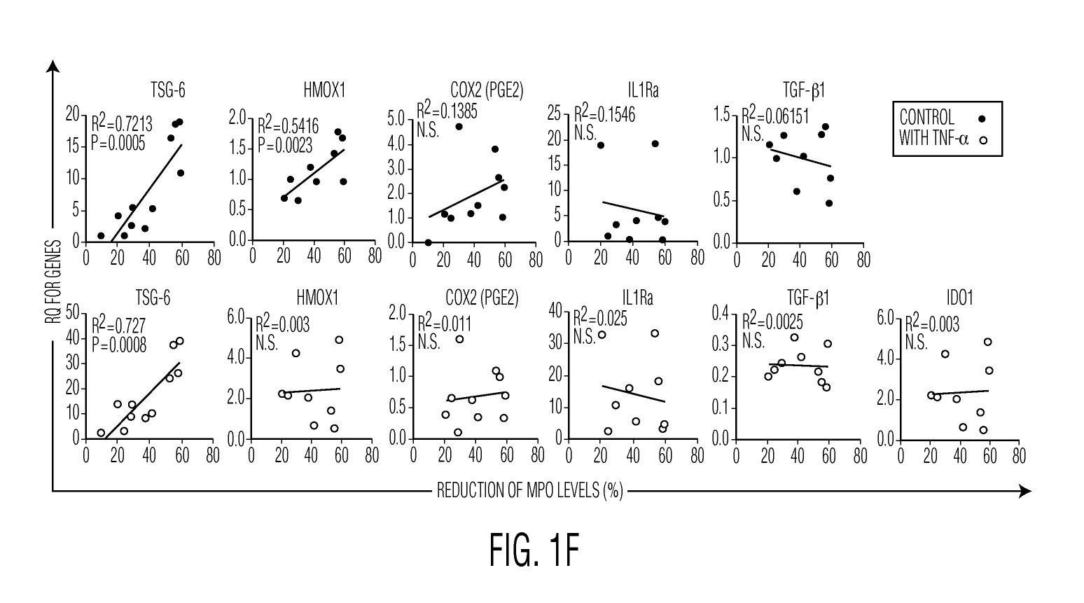

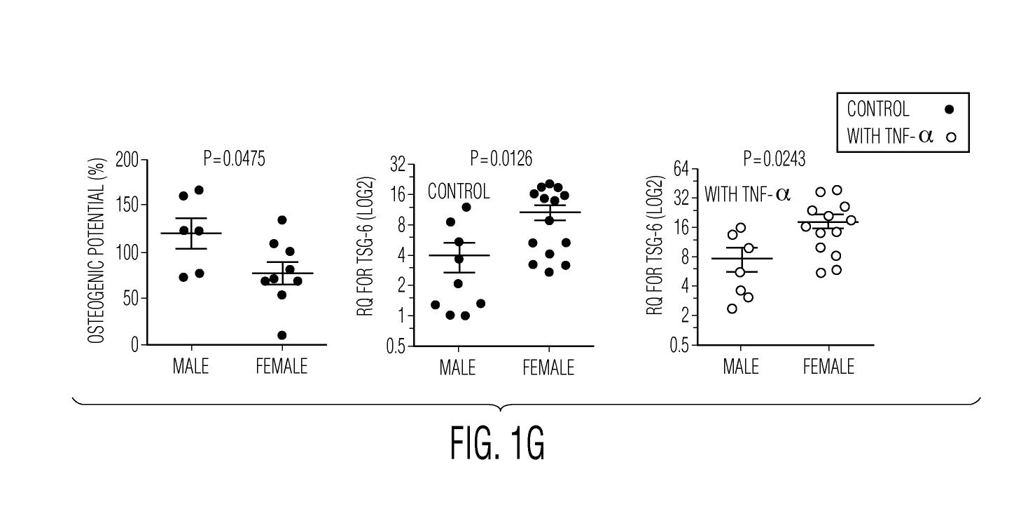

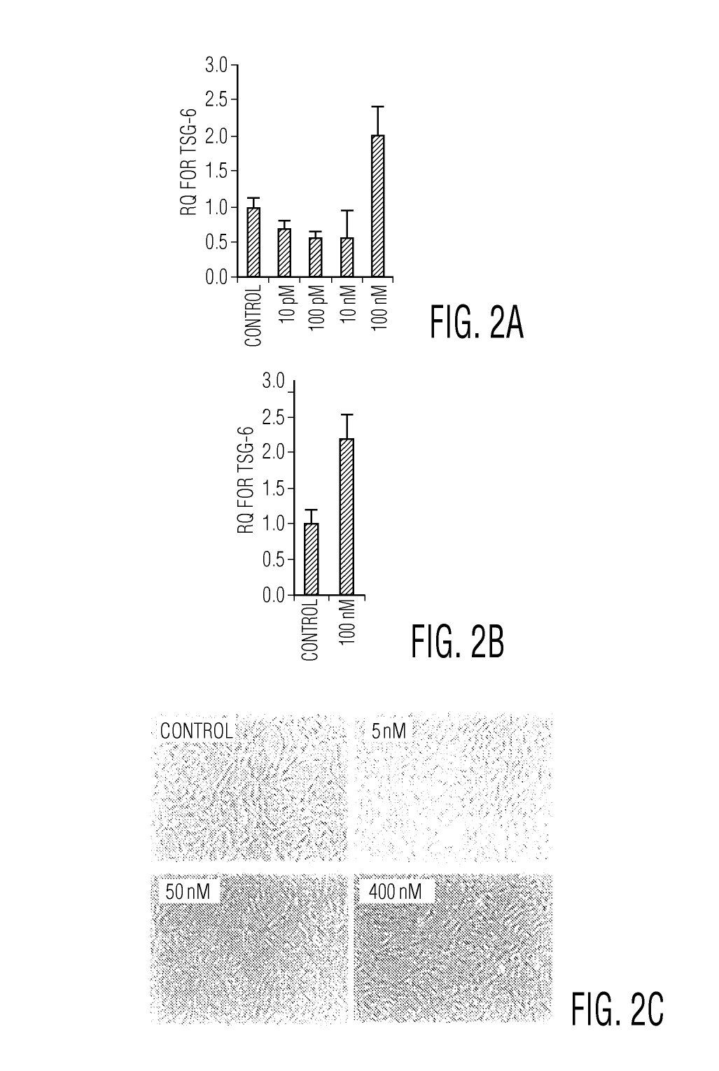

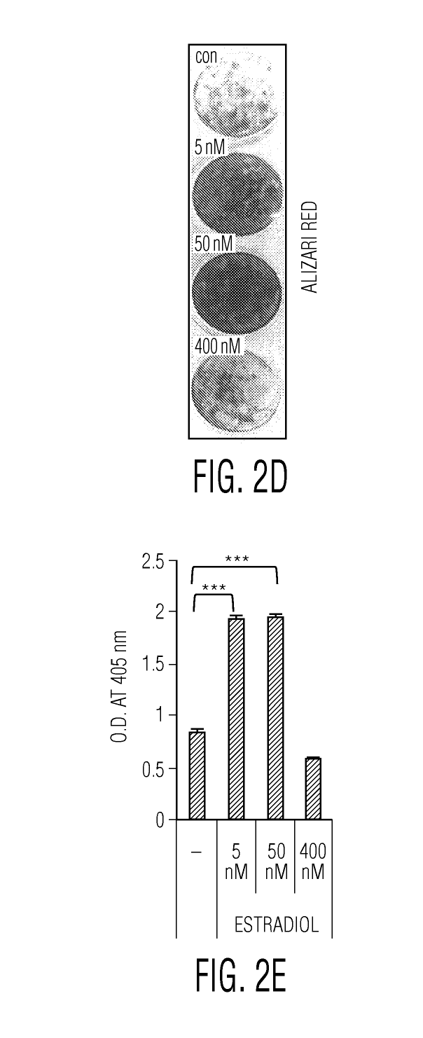

Of special interest was that assays on a small cohort suggested that hMSCs from female donors were more effective in suppressing inflammation in the cornea than hMSCs from male donors (FIG. 1D). There was also a negative correlation with height (FIG. 1D) and weight of donors (FIG. 1D) that may or may not have reflected the gender difference. To a lesser degree, the gender differences were observed in comparisons of osteogenic differentiation and the levels of TSG-6 mRNA in the cells (FIG. 1G). In order to explore further the apparent gender bias, we examined the effects of incubating hMSCs with estradiol, the female hormone that reaches the highest peak values in serum (up to 1.6 nM) during the menstrual cycle (Kratz, 2004). One-day exposure of hMSCs to low doses of estradiol decreased TSG-6 levels in hMSCs, whereas a high-dose estradiol increased TSG-6 in hMSCs (FIG. 2A). The effects of the high-dose persisted after incubation with 100 nM for 2 days, and the increased levels of TSG-6 mRNA were observed after the cells were incubated without estradiol for an additional 2 days (FIG. 2B). Furthermore, pre-treatment of low-doses of estradiol in hMSCs for 4 days promoted osteogenic differentiation, whereas pre-treatment with a high-dose of 400 nM for 4 days suppressed osteogenic differentiation (FIG. 2D and E) without affecting cell viability (FIG. 2C). The results suggested that the periodic bursts of female hormones during menstruation could contribute to but not account fully for the differences between male and female hMSCs. It is of interest that the data suggesting a gender bias in donors of hMSCs is consistent with a large body of literature demonstrating marked differences in susceptibility to diseases between men and women (Verdonk, 2012). The differences observed here between hMSCs were maintained during expansion of the cells in culture under the same conditions and could be explained by relatively long-term effects of the cycles of inflammation and hormonal bursts that occur during menstruation (Martin-Millan, 2013, Evans, 2012).

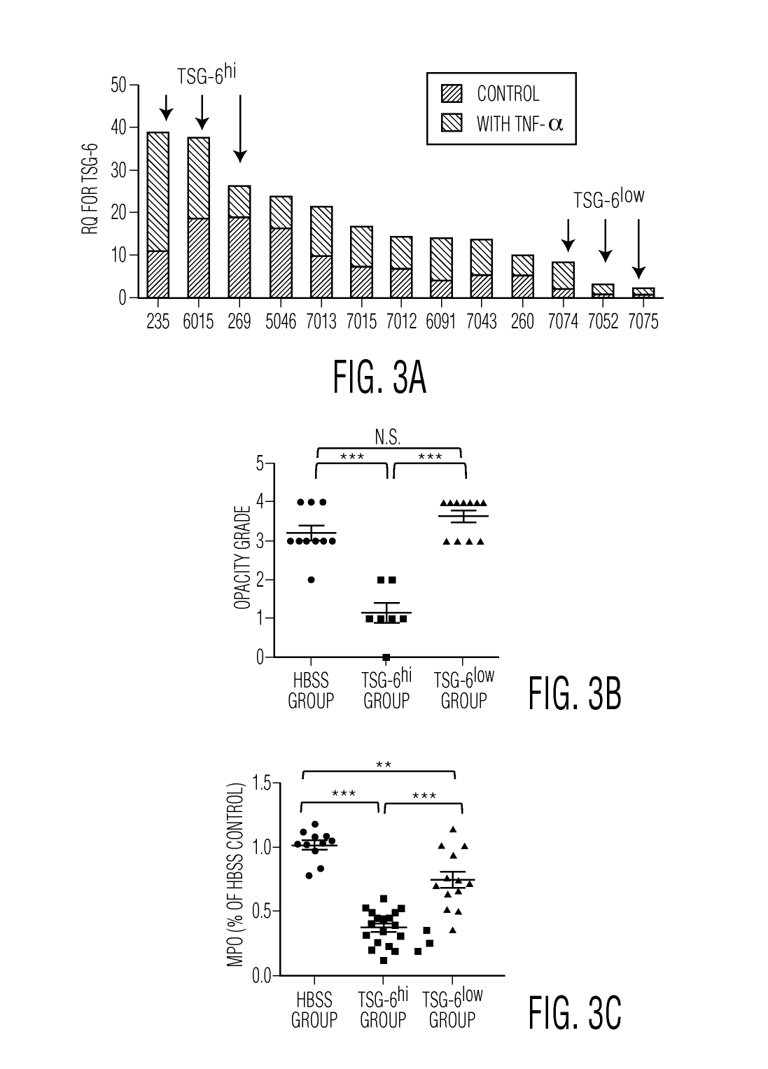

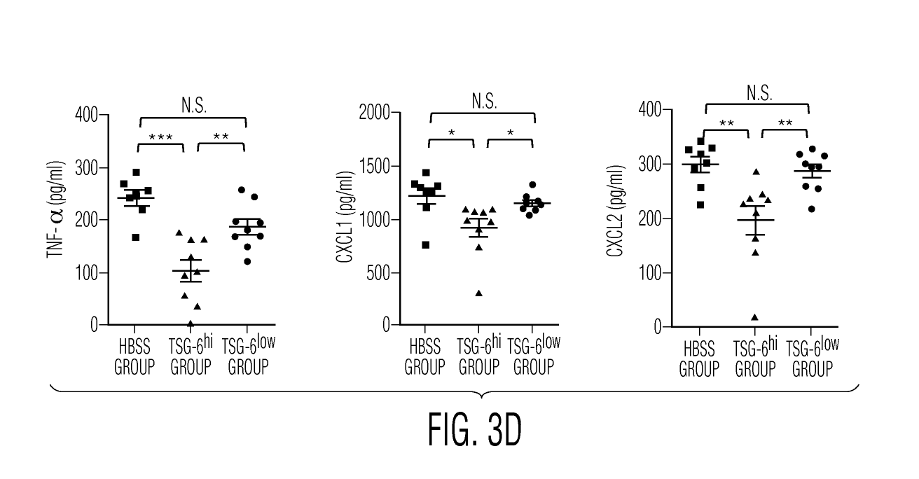

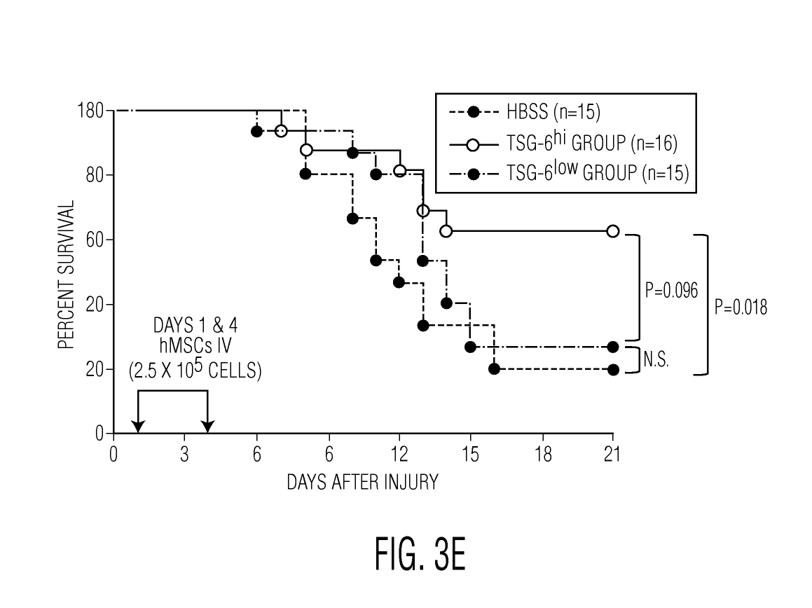

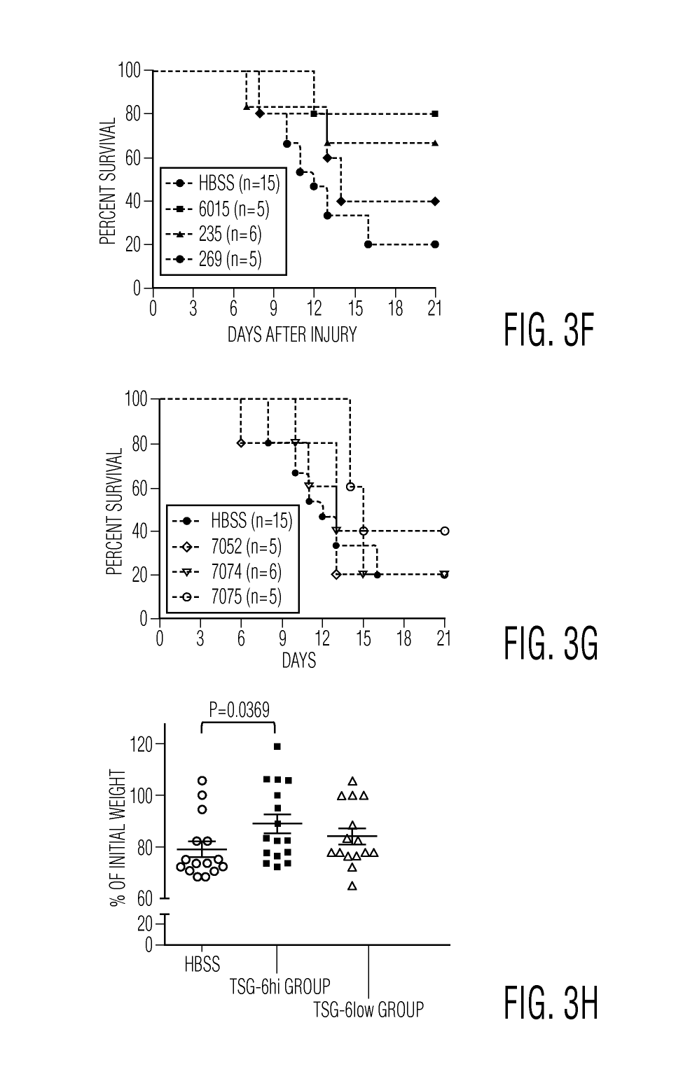

In order to evaluate the biomarker that predicts efficacy of hMSCs in three in vivo models for sterile inflammation, we defined three effective donors of the hMSCs as TSG-6^{hi} and three ineffective donors of the hMSCs as TSG-6^{low} (FIG. 3A). The TSG-6^{hi} hMSC group compared to the TSG-6^{low} hMSC group was more effective both in preventing corneal opacity and in decreasing the inflammation as indicated by the MPO levels (FIG. 3B and C). In a mouse model for peritonitis (Choi, 2011), IP injection of TSG-6^{hi} hMSCs but not TSG-6^{low} MSCs decreased pro-inflammatory cytokines in peritoneum lavage (FIG. 3D). In a bleomycin-induced lung injury mouse model (Foskett, 2014), IV administration of TSG-6^{hi} hMSCs but not TSG-6^{low} MSCs improved survival (FIG. 3E) and preserved body weight in the mice (FIG. 3F) compared to a control group. The differences between mice that received TSG-6^{hi} hMSCs and TSG-6^{low} MSCs, however, were not significant because one (donor 265 and donor 7075) of each group showed moderate survival (FIG. 3G). The more variable results in the bleomycin model probably reflect the complexity of this model in which bleomycin triggers apoptosis and releases oxidants, and this followed first by a phase marked by invasion of inflammatory and immune cells and then by a fibrotic phase (Hay, 1991).

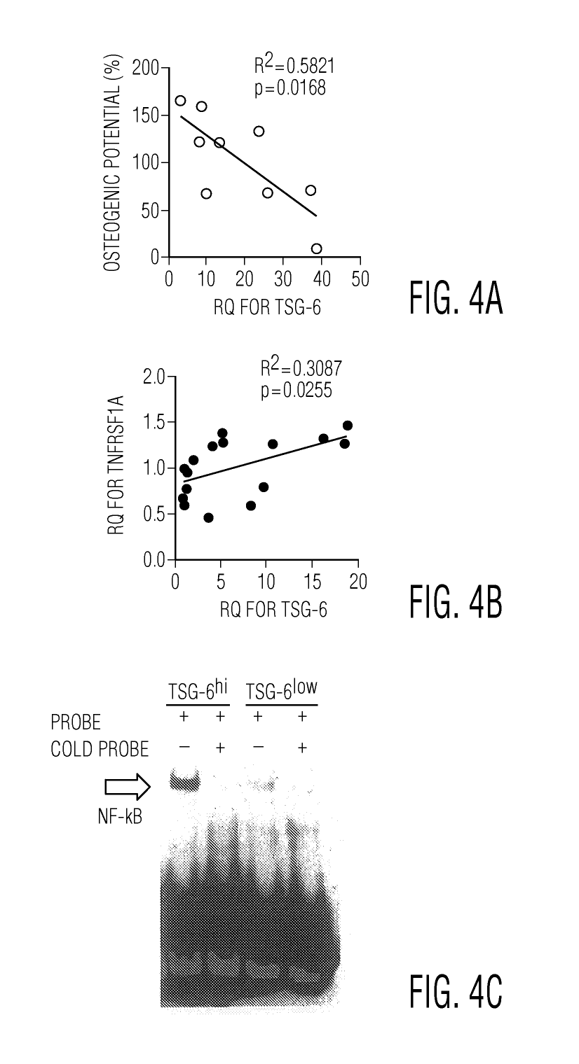

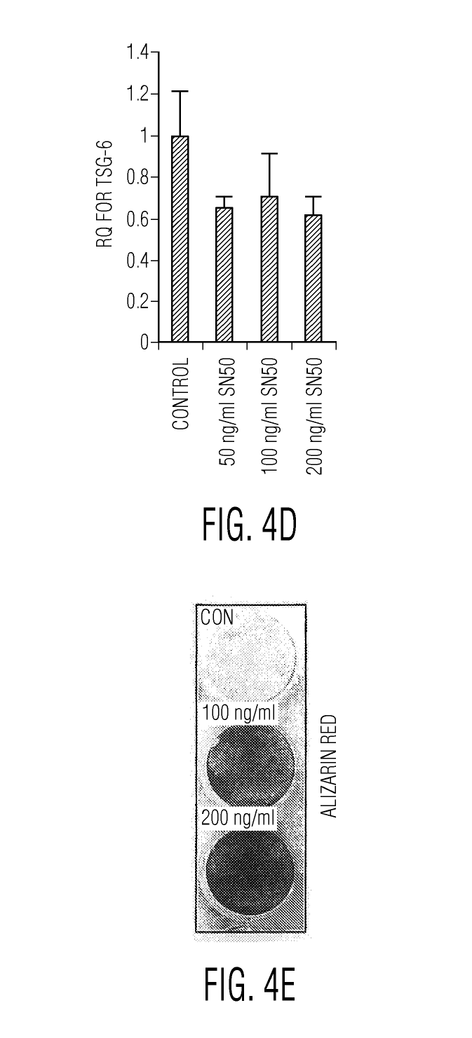

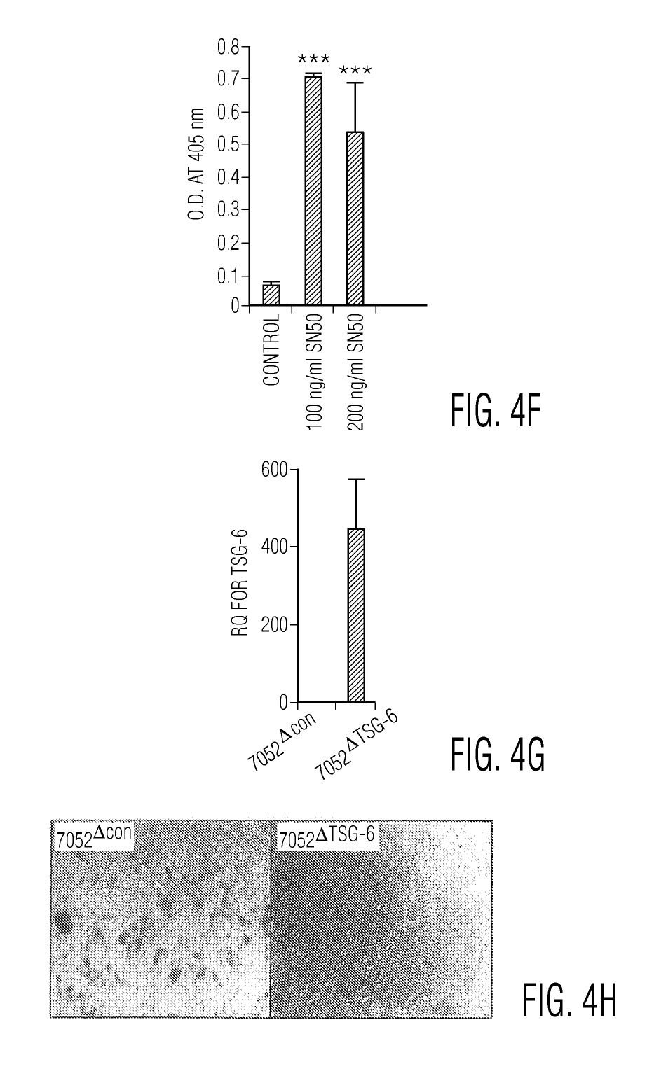

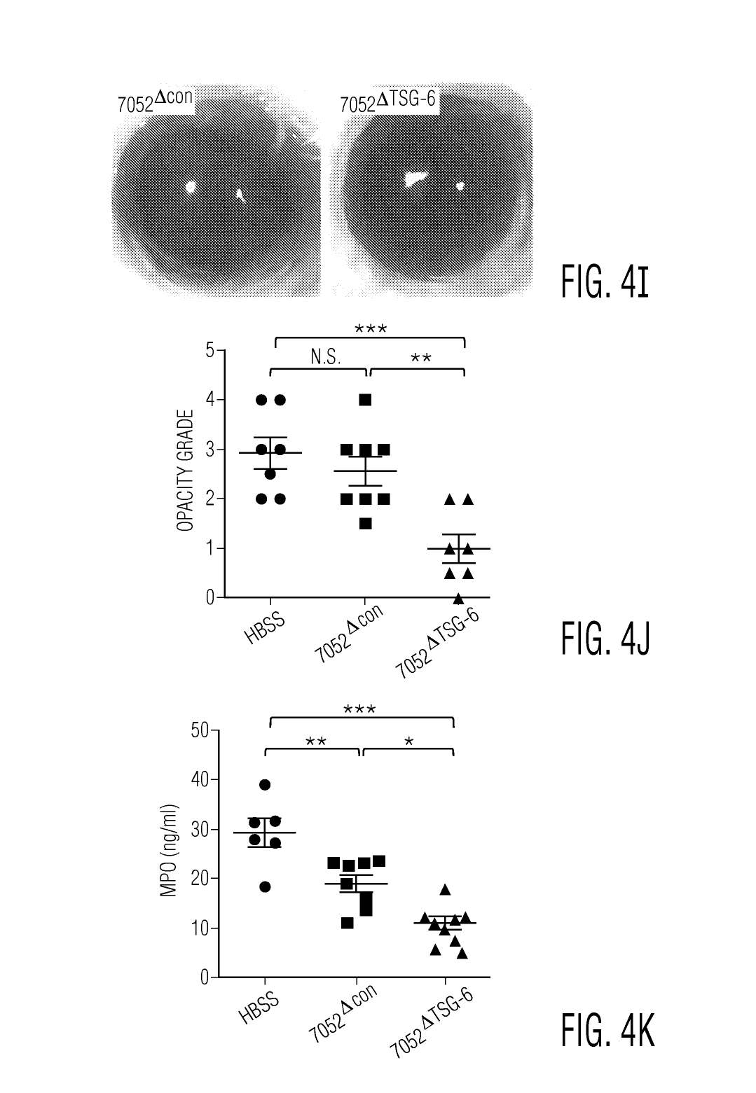

The levels of TSG-6 mRNA showed a negative correlation with the potential for osteogenic differentiation (FIG. 4A). Recently, the NF-κB signal transduction pathway was implicated as a negative regulator of osteoblastic differentiation and suppression of this pathway increased osteoblastic differentiation and mineralization in vitro (Yamaguchi, 2009). Since TSG-6 is a TNFα-stimulated gene (Klampfer, 1995), and involvement of NE-κB signaling was suggested by the slightly positive correlation between the levels of mRNA for TSG-6 and TNFRSF1A, tumor necrosis factor receptor superfamily member 1A (FIG. 4B), we examined NF-κB activation in the nuclear extracts of hMSCs by EMSA assays. As we expected, NF-κB binding activity was present at very low levels in TSG-6^{low} group but at higher levels in TSG-6^{hi} group (FIG. 4C). When NF-κB activity was inhibited by SN50 (Kolenko, 1999), the levels of TSG-6 mRNA were decreased (FIG. 4D). Also, pre-treatment of hMSCs with SN-50 for 4 days increased the potential of the cells to differentiate into osteoblasts (FIG. 4E and F). In addition, we over-expressed TSG-6 in male hMSCs with a low level of expression of the gene (Donor 7052) (FIG. 4G). Over-expression of TSG-6 decreased the potential of the cells to differentiate into osteoblasts (FIG. 4H) and increased the effectiveness of the hMSCs in decreasing the opacity and the MPO levels of the cornea model (FIGS. 4I to K).

### Discussion

The use of TSG-6 as a biomarker for efficacy of hMSCs in suppressing inflammation in vivo is consistent with our previous observations. It is a naturally occurring protein of 35 kDa that is secreted by most cells in response to pro-inflammatory cytokines and it has multiple actions that are linked to modulation of inflammation and stabilization of the extracellular matrix. Among its multiple actions is that TSG-6 either directly or through a complex with hyaluronan, binds to CD44 on resident macrophages in a manner that decreases TLR/NF-kB signaling and modulates the initial phase of the inflammatory response of most tissues. (Choi, 2011; Kota, 2013). hMSCs were observed to lose their effectiveness in several animal models for human diseases after siRNAs were used to knock down expression of TSG-6 (Lee, 2009; Roddy, 2011; Kota, 2013; Choi, 2011; Oh, 2012). Also, administration of recombinant TSG-6 reproduced most of the beneficial effects of the hMSCs (Lee, 2009; Roddy, 2011; Kota, 2013; Foskett, 2014; Oh, 2012; Choi, 2011). The role of TSG-6 in the cornea model was validated here further by the demonstration that over-expression of TSG-6 enhanced greatly the effectiveness of hMSCs. The data to date, however, have not established that TSG-6 is the only paracrine factor secreted by MSCs that suppresses inflammation, and it is possible that genes expressed upstream of TSG-6 may prove to be useful biomarkers.

One of the critical observations was that the conventional assays used to characterize hMSCs did not predict the efficacy of the cells in suppressing inflammation in vivo. Also, there was no significant correlation with expression of several other genes linked previously to the therapeutic potentials of the cells. One important exception was a highly negative correlation between the effectiveness of the cells in suppressing inflammation in the cornea model and their potential for osteogenic differentiation in culture. The negative correlation with osteogenic differentiation provided an independent validation for the differences among hMSC donors. The negative correlation with osteogenic differentiation suggests that hMSCs optimal for one application, such as suppression of inflammation, may be sub-optimal for other applications, such as bone engineering.

The results presented here may overcome a major barrier to research with hMSCs: they provide the first biomarker that can predict the efficacy of the hMSCs in producing therapeutic effects in sterile inflammation disease models. Assays in the model for chemical injury of the cornea demonstrated marketed differences in the inflammation-suppressive efficacy of different preparations of hMSCs, here defined by the donors that provided the bone marrow aspirates. We demonstrated that the levels of mRNA for TSG-6 in the hMSCs predicted their efficacy in the cornea model as well as in a model for zymosan-induced peritonitis and, with somewhat less accuracy, in a more complex model of bleomycin-induced lung injury. The RT-PCR assay for TSG-6 that was employed is robust and it can be performed in about 4 hours. Therefore the levels of expression of TSG-6 with this assay should be useful in selecting hMSCs to reduce the variability in experiments and clinical trials with MSCs for the large number of diseases in which sterile inflammation is now recognized to play a critical role. (Prockop, 2012; Lee, 2009; Chen, 2010; Okin, 2012).

The disclosures of all patents, publications (including published patent applications), depository accession numbers, and database accession numbers hereby are incorporated by reference to the same extent as if each patent, publication, depository accession number, and database accession number were incorporated individually by reference.

It is to be understood, however, that the scope of the present invention is not to be limited to the specific embodiments described above. The invention may be practiced other than as particularly described and still be within the scope of the accompanying claims.

### REFERENCE LIST

Bernardo, M. E., and Fibbe, W. E. (2013). Mesenchymal stromal cells: sensors and switchers of inflammation. Cell. Stem Cell. 13, 392-402.
Chen, G. Y., and Nunez, G. (2010). Sterile inflammation: sensing and reacting to damage. Nat. Rev. Immunol. 10, 826-837.
Choi, H., Lee, R. H., Bazhanov, N., Oh, J. Y., and Prockop, D. J. (2011). Anti-inflammatory protein TSG-6 secreted by activated MSCs attenuates zymosan-induced mouse peritonitis by decreasing TLR2/NF-kappaB signaling in resident macrophages. Blood 118, 330-338.
Colter, D. C., Sekiya, I., and Prockop, D. J. (2001). Identification of a subpopulation of rapidly self-renewing and multipotential adult stem cells in colonies of human marrow stromal cells. Proc. Natl. Acad. Sci. U. S. A. 98, 7841-7845.
Digioralmo, C. M., et al., Propagation and senescence of human marrow stromal cells in culture: a simple colony-forming assay identified samples with the greatest potential to propagate and differentiate. Br. J. Haematol. 107, 275-281 (1999).
English, K. Mechanisms of mesenchymal stromal cell immmunomodulation. Immunol. Cell. Biol. 91, 19-26 (2013).
Evans, J., and Salamonsen, L. A. (2012). Inflammation, leukocytes and menstruation. Rev. Endocr Metab. Disord. 13, 277-288.
Foskett, A. M., et al. Phase-directed therapy: TSG-6 targeted to early inflammation improves bleomycin-injured lungs. Am. J. Physiol. Lung Cell. Mol. Physiol. 306, L120-131 (2014).
Gregory, C. A., Gunn, W. G., Peister, A., and Prockop, D. J. (2004). An Alizarin red-based assay of mineralization by adherent cells in culture: comparison with cetylpyridinium chloride extraction. Anal. Biochem. 329, 77-84.
Hay, J., Shahzeidi, S., and Laurent, G. Mechanisms of bleomycin-induced lung damage. Arch. Toxicol. 65, 81-94 (1991).
Huang, S., Feng, C., Wu, Y., Yang, S., Ma, K., Wu, X., and Fu, X. (2013). Dissimilar characteristics of umbilical cord mesenchymal stem cells from donors of different ages. Cell Tissue Bank. 14, 707-713.
Keating, A. (2012). Mesenchymal stromal cells: new directions. Cell. Stem Cell. 10, 709-716.
Klampfer, L., Chen-Kiang, S., & Vilcek, J. Activation of the TSG-6 gene by NF-IL6 requires two adjacent NF-IL6 binding sites. J. Biol. Chem. 270, 3677-3682 (1995).
Kolenko, V., Bloom, T., Rayman, P., Bukowski, R., Hsi, E., and Finke, J. (1999). Inhibition of NF-kappa B activity in human T lymphocytes induces caspase-dependent apoptosis without detectable activation of caspase-1 and -3. J. Immunol. 163, 590-598.
Kota, D. J., Wiggins, L.L., Yoon, N., and Lee, R. H. (2013). TSG-6 produced by hMSCs delays the onset of autoimmune diabetes by suppressing Th1 development and enhancing tolerogenicity. Diabetes. 62, 2048-2058.
Kratz, A., Ferraro, M., Sluss, P. M., and Lewandrowski, K. B. (2004). Case records of the Massachusetts General Hospital. Weekly clinicopathological exercises. Laboratory reference values. N. Engl. J. Med. 351, 1548-1563.
Lee, R. H., Oh, J. Y., Choi, H., and Bazhanov, N. (2011). Therapeutic factors secreted by mesenchymal stromal cells and tissue repair. J. Cell. Biochem. 112, 3073-3078.
Lee, R. H., Pulin, A. A., Seo, M. J., Kota, D. J., Ylostalo, J., Larson, B. L., Semprun-Prieto, L., Delafontaine, P., and Prockop, D. J. (2009). Intravenous hMSCs improve myocardial infarction in mice because cells embolized in lung are activated to secrete the anti-inflammatory protein TSG-6. Cell. Stem Cell. 5, 54-63.
Martin-Millan, M., and Castaneda, S. (2013). Estrogens, osteoarthritis and inflammation. Joint Bone Spine 80, 368-373.
Meisel, et al. Human bone marrow stromal cells inhibit allogeneic T-cell responses by indoleamine 2,3-dioxygenase-mediated tryptophan degradation. Blood 103, 4619-4621 (2004).
Milner, C. M., and Day, A. J. (2003). TSG-6: a multifunctional protein associated with inflammation. J. Cell. Sci. 116, 1863-1873.
Nemeth, K., et al. Bone marrow stromal cells attenuate sepsis via prostaglandin E(2)-dependent reprogramming of host macrophages to increase their Interleukin-10 production Nat. Med. 15, 42-49 (2009).
Oh, J. Y., Lee, R. H., Yu, J. M., Ko, J. H., Lee, H. J., Ko, A. Y., Roddy, G. W., and Prockop, D. J. (2012). Intravenous mesenchymal stem cells prevented rejection of allogeneic corneal transplants by aborting the early inflammatory response. Mol. Ther. 20, 2143-2152.
Oh, J. Y., Roddy, G. W., Choi, H., Lee, R. H., Ylostalo, J. H., Rosa, R. H.,Jr, and Prockop, D. J. (2010). Anti-inflammatory protein TSG-6 reduces inflammatory damage to the cornea following chemical and mechanical injury. Proc. Natl. Acad. Sci. U. S. A. 107, 16875-16880.
Okin, D., and Medzhitov, R. (2012). Evolution of inflammatory diseases. Curr. Biol. 22, R733-40.
Ortiz, L. A., et al. Interleukin 1 receptor antagonist mediates the anti-inflammatory and antifibrotic effect of mesenchymal stem cells during lung injury. Proc. Nat. Acad. Sci. USA, 104. 11002-11007 (2007).
Owen, M., and Friedenstein, A. J. (1988). Stromal stem cells: marrow-derived osteogenic precursors. Ciba found. Symp. 136, 42-60.
Phinney, D. G., Kopen, G., Righter, W., Webster, S., Tremain, N., and Prockop, D. J. (1999). Donor variation in the growth properties and osteogenic potential of human marrow stromal cells. J. Cell. Biochem. 75, 424-436.
Prockop, D. J., and Oh, J. Y. (2012). Medical therapies with adult stem/progenitor cells (MSCs): a backward journey from dramatic results in vivo to the cellular and molecular explanations. J. Cell. Biochem. 113, 1460-1469.
Prockop, D. J., and Oh, J. Y. (2012). Mesenchymal stem/stromal cells (MSCs): role as guardians of inflammation. Mol. Ther. 20, 14-20.
Roddy, G. W., Oh, J. Y., Lee, R. H., Bartosh, T. J., Ylostalo, J., Coble, K., Rosa, R. H., Jr, and Prockop, D. J. (2011). Action at a distance: systemically administered adult stem/progenitor cells (MSCs) reduce inflammatory damage to the cornea without engraftment and primarily by secretion of TNF-alpha stimulated gene/protein 6. Stem Cells 29, 1572-1579.
Sekiya, I., et al., Expansion of human adult stem cells from bone marrow stroma: conditions that maximize the yields of early progenitors and evaluate their quality. Stem Cells 20, 530-541 (2002).

Syed, B. A., and Evans, J. B. (2013). Stem cell therapy market. Nat. Rev. Drug Discov. 12, 185-186.

Verdonk, P., and Klinge, I. (2012). Mainstreaming sex and gender analysis in public health genomics. Gend. Med. 9, 402-410.
Wisniewski, H. G., Snitkin, E. S., Mindrescu, C., Sweet, M. H., and Vilcek, J. (2005). TSG-6 protein binding to glycosaminoglycans: formation of stable complexes with hyaluronan and binding to chondroitin sulfates. J. Biol. Chem. 280, 14476-14484.
Wisniewski, H. G., and Vilcek, J. (1997). TSG-6: an IL-1/TNF-inducible protein with anti-inflammatory activity. Cytokine Growth Factor Rev. 8, 143-156.

### Wu, et al., Methods in Gene Biotechnology, CRC Press (1997) pgs. 16-21.

Yamaguchi, M., and Weitzmann, M.N. (2009). The estrogen 17beta-estradiol and phytoestrogen genistein mediate differential effects on osteoblastic NF-kappaα activity. Int. J. Mol. Med. 23, 297-301.

What is claimed is:

1. A composition comprising isolated mesenchymal stem cells that produce mRNA encoding tumor necrosis factor-6 gene (TSG-6) protein or a biologically active fragment, derivative, or analogue thereof in an amount of at least a preselected amount, as measured by an assay which comprises:
assaying the level of mRNA encoding TSG-6 protein or a biologically active fragment, derivative, or analogue thereof that is produced by a population of isolated mesenchymal stem cells; and
determining, from the level of mRNA encoding TSG-6 protein or a biologically active fragment, derivative, or analogue thereof produced by said population of isolated mesenchymal stem cells, whether said population of isolated mesenchymal stem cells produces mRNA encoding TSG-6 protein or a biologically active fragment, derivative, or analogue thereof in an amount of said at least preselected amount.

2. The composition of claim 1 wherein said assaying for said levels of mRNA produced by said population of isolated mesenchymal stem cells is conducted by RT-PCR.

3. A process for preparing a composition comprising isolated mesenchymal stem cells that produce mRNA encoding tumor necrosis factor-α stimulating gene 6 (TSG-6) protein or a biologically active fragment, derivative, or analogue thereof in an amount of at least a preselected amount, comprising:
(a) providing a population of isolated mesenchymal stem cells;
(b) assaying the level of mRNA encoding TSG-6 protein or a biologically active fragment, derivative, or analogue thereof that is produced by said population of isolated mesenchymal stem cells; and
(c) determining, from the level of mRNA encoding TSG-6 protein or a biologically active fragment, derivative, or analogue thereof produced by said population of isolated mesenchymal stem cells, whether said population of isolated mesenchymal stem cells produces mRNA encoding TSG-6 protein or a biologically active fragment, derivative, or analogue thereof in an amount of at least said preselected amount.

4. The process of claim 3 wherein said assaying for said levels of mRNA produced by said population of isolated mesenchymal stem cells is conducted by RT-PCR.

5. A composition comprising isolated mesenchymal stem cells that produce mRNA encoding tumor necrosis factor-α stimulating gene 6 (TSG-6) protein or a biologically active fragment, derivative, or analogue thereof in an amount that does not exceed a preselected amount, as measured by an assay which comprises:
assaying the level of mRNA encoding TSG-6 protein or a biologically active fragment, derivative, or analogue thereof that is produced by a population of isolated mesenchymal stem cells; and
determining, from the level of mRNA encoding TSG-6 protein or a biologically active fragment, derivative, or analogue thereof produced by said population of isolated mesenchymal stem cells, whether said isolated mesenchymal stem cells produce mRNA encoding TSG-6 protein or a biologically active fragment, derivative, or analogue thereof in an amount that does not exceed said preselected amount.

6. The composition of claim 5 wherein said assaying for said levels of mRNA produced by said population of isolated mesenchymal stem cells is conducted by RT-PCR.

7. A process for preparing a composition comprising isolated mesenchymal stem cells that produce mRNA encoding tumor necrosis factor-α stimulating gene 6 (TSG-6) protein or a biologically active fragment, derivative, or analogue thereof in an amount that does not exceed a preselected amount, comprising:
(a) providing a population of isolated mesenchymal stem cells;
(b) assaying the level of mRNA encoding TSG-6 protein or a biologically active fragment, derivative, or analogue thereof that is produced by said population of isolated mesenchymal stem cells; and
(c) determining from the level of mRNA encoding TSG-6 protein or a biologically active fragment, derivative, or analogue thereof produced by said population of isolated mesenchymal stem cells, whether said population of isolated mesenchymal stem cells produces mRNA encoding TSG-6 protein or a biologically active fragment, derivative, or analogue thereof in an amount that does not exceed said predetermined amount.

8. The process of claim 7 wherein said assaying for said levels of mRNA produced by said population of isolated mesenchymal stem cells is conducted by RT-PCR.

9. A method of stimulating isolated mesenchymal stem cells to express increased amounts of tumor necrosis factor-α gene 6 (TSG-6) protein or a biologically active fragment, derivative, or analogue thereof comprising:
contacting said isolated mesenchymal stem cells with at least one female hormone or derivative or analogue thereof in an amount of at least 50 nM, whereby said isolated mesenchymal stem cells express TSG-6 protein or a biologically active fragment, derivative, or analogue thereof in an amount greater than the amount of TSG-6 protein or a biologically active fragment, derivative, or analogue thereof expressed by said isolated mesenchymal stem cells prior to the contacting of said isolated mesenchymal stem cells with said at least one female hormone or derivative or analogue thereof in an amount of at least 50 nM.

10. The method of claim 9 wherein said at least one female hormone or derivative or analogue thereof is estradiol.

11. The method of claim 9 wherein said isolated mesenchymal stem cells are contacted with said at least one female hormone or derivative or analogue thereof in an amount of at least 100 nM.

12. Isolated mesenchymal stem cells prepared by the method of claim 9.

13. A method of treating an inflammatory disease or disorder in an animal comprising:
administering to said animal the isolated mesenchymal stem cells of claim 12 in an amount effective to treat said inflammatory disease or disorder in said animal.

14. The method of claim 13 wherein said animal is a primate.

15. The method of claim 14 wherein said primate is a human.

16. A kit for determining the presence and/or amount of an RNA sequence encoding TSG-6 protein or a biologically active fragment, derivative, or analogue thereof in mesenchymal stem cells, comprising:
(a) a population of mesenchymal stem cells that produce a predetermined amount of an RNA sequence encoding TSG-6 protein or a biologically active fragment, derivative, or analogue thereof;
(b) at least two identical culture media for culturing and expanding mesenchymal stem cells;
(c) instructions for culturing and expanding said mesenchymal stem cells;
(d) at least two identical sets of reagents for extracting RNA from said mesenchymal stem cells;
(e) instructions for extracting RNA from said mesenchymal stem cells;
(f) at least three microplates suitable for conducting reverse transcription PCR of RNA;
(g) a predetermined amount of an RNA sequence encoding TSG-6 protein or a biologically active fragment, derivative, or analogue thereof, said predetermined amount of at least one RNA sequence encoding a protein having been extracted previously from said mesenchymal stem cells of step (a);
(h) a 3′ DNA primer and a 5′ DNA primer corresponding to the RNA sequence encoding TSG-6 protein or a biologically active fragment, derivative, or analogue thereof of which the presence and/or the amount thereof is to be determined;
(i) at least two identical sets of reagents for conducting reverse transcription PCR;
(j) instructions for conducting reverse transcription PCR of RNA;
(k) means for determining the presence and/or amount of the RNA sequence encoding TSG-6 protein or a biologically active fragment, derivative, or analogue thereof; and
(l) instructions for determining the presence and/or amount of the RNA sequence encoding TSG-6 protein or a biologically active fragment, derivative, or analogue thereof.


SEQUENCE LISTING (3 sequences)

SEQ ID NO: 1 - protein, 277 aa, Homo sapiens
  TSG-6 protein
MIILIYLFLLLWEDTQGWGFKDGIFHNSIWLERAAGVYHREARSGKYKLTYAEAKAVCEFEGGHLATYKELEAARKIGFHVCAAGWMAKGRVGYPIVKPGPNCGFGKTGIIDYGIRLNRSERWDAYCYNPHAKECGGVFTDPKEIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 2 - DNA, 29 bp, Artificial sequence
  PCR primer
cggggtaccatgatcatcttaatttactt

SEQ ID NO: 3 - DNA, 24 bp, Artificial sequence
  PCR primer
ggtgatcagtggctaaatcttcca